SEQUENCE LISTING (34 sequences)

SEQ ID NO: 1 - protein, 10 aa, Homo sapiens
KGAIIGLMVG

SEQ ID NO: 2 - protein, 15 aa, Homo sapiens
GSNKGAIIGLMVGGV

SEQ ID NO: 3 - protein, 12 aa, Homo sapiens
VFFAEDVGSNKG

SEQ ID NO: 4 - protein, 12 aa, Homo sapiens
AEDVGSNKGAII

SEQ ID NO: 5 - protein, 12 aa, Homo sapiens
VGSNKGAIIGLM

SEQ ID NO: 6 - protein, 12 aa, Homo sapiens
NKGAIIGLMVGG

SEQ ID NO: 7 - protein, 28 aa, Homo sapiens
QKLVFFAEDVGSNKGAIIGLMVGGVVIA

SEQ ID NO: 8 - protein, 20 aa, Homo sapiens
QKLVFFAEDVGSNKGAIIGL

SEQ ID NO: 9 - protein, 10 aa, Homo sapiens
AEDVGSNKGA

SEQ ID NO: 10 - protein, 15 aa, Homo sapiens
LVFFAEDVGSNKGAI

SEQ ID NO: 11 - protein, 15 aa, Homo sapiens
VFFAEDVGSNKGAII

SEQ ID NO: 12 - protein, 10 aa, Homo sapiens
VFFAEDVGSN

SEQ ID NO: 13 - protein, 15 aa, Homo sapiens
QKLVFFAEDVGSNKG

SEQ ID NO: 14 - protein, 15 aa, Homo sapiens
AEDVGSNKGAIIGLM

SEQ ID NO: 15 - protein, 15 aa, Homo sapiens
KLVFFAEDVGSNKGA

SEQ ID NO: 16 - protein, 42 aa, Homo sapiens [sequence omitted]

SEQ ID NO: 17 - protein, 14 aa, Homo sapiens
DAEFRHDSGYEVHH

SEQ ID NO: 18 - protein, 17 aa, Homo sapiens
KLVFFAEDVGSNKGAII

SEQ ID NO: 19 - protein, 16 aa, Homo sapiens
LVFFAEDVGSNKGAII

SEQ ID NO: 20 - protein, 15 aa, Homo sapiens
EDVGSNKGAIIGLMV

SEQ ID NO: 21 - protein, 15 aa, Homo sapiens
KGAIIGLMVGGVVIA

SEQ ID NO: 22 - protein, 12 aa, Homo sapiens
QKLVFFAEDVGS

SEQ ID NO: 23 - protein, 12 aa, Homo sapiens
KLVFFAEDVGSN

SEQ ID NO: 24 - protein, 12 aa, Homo sapiens
LVFFAEDVGSNK

SEQ ID NO: 25 - protein, 12 aa, Homo sapiens
FFAEDVGSNKGA

SEQ ID NO: 26 - protein, 12 aa, Homo sapiens
FAEDVGSNKGAI

SEQ ID NO: 27 - protein, 12 aa, Homo sapiens
EDVGSNKGAIIG

SEQ ID NO: 28 - protein, 12 aa, Homo sapiens
DVGSNKGAIIGL

SEQ ID NO: 29 - protein, 12 aa, Homo sapiens
GSNKGAIIGLMV

SEQ ID NO: 30 - protein, 12 aa, Homo sapiens
SNKGAIIGLMVG

SEQ ID NO: 31 - protein, 12 aa, Homo sapiens
KGAIIGLMVGGV

SEQ ID NO: 32 - protein, 12 aa, Homo sapiens
GAIIGLMVGGVV

SEQ ID NO: 33 - protein, 12 aa, Homo sapiens
AIIGLMVGGVVI

SEQ ID NO: 34 - protein, 12 aa, Homo sapiens
IIGLMVGGVVIA

(19) United States
(12) Patent Application Publication
(10) Pub. No.: US 2011/0178024 A1
(43) Pub. Date: Jul. 21, 2011

(54) AMYLOID BETA-PEPTIDES AND METHODS OF USE THEREOF

(75) Inventor: Alon Monsonego, Moshav Nir-Banim (IL)

(73) Assignee: Ben Gurion University of the Negev Research and Development Authority, Beer-Sheva (IL)

(21) Appl. No.: 13/121,421

(22) PCT Filed: Sep. 24, 2009
(86) PCT No.: PCT/IL09/00922

§ 371 Date: Mar. 29, 2011

Related U.S. Application Data
(60) Provisional application No. 61/136,729, filed on Sep. 29, 2008.

(51) Int. Cl. A61K 38/10; G01N 33/50; C07K 7/08; A61P 25/28

(52) U.S. Cl. 514/17.8; 435/7.21; 530/327; 514/21.5

(57) ABSTRACT

The present invention relates to polypeptides, compositions, and methods of use thereof for optimized treatment of diseases or disorders associated with amyloid beta protein (Aβ) accumulation in a subject. This invention also relates to polypeptides, compositions, and methods of use thereof for optimized immunization against diseases characterized by Aβ accumulation in a subject. This invention further relates to pharmaceutical formulations comprising these polypeptides and methods of use for administering to a subject an optimized Aβ peptide that elicits a beneficial T cell response; for example, a T cell response reducing Aβ accumulation in the brain of a subject, without causing an encephalitic response.

### FIELD OF INVENTION

This invention provides polypeptides, compositions, and methods of use thereof for optimized treatment of diseases or disorders associated with amyloid beta protein (Aβ) accumulation in a subject. This invention further provides polypeptides, compositions, and methods of use thereof for optimized immunization against diseases characterized by Aβ accumulation in a subject.

### BACKGROUND OF THE INVENTION

Alzheimer's disease (AD), first described by the Bavarian psychiatrist Alos Alzheimer in 1907, is a progressive neurological disorder that begins with short-term memory loss and proceeds to disorientation, impairment of judgment and reasoning and, ultimately, dementia. The course of the disease usually leads to death in a severely debilitated, immobile state between four and 12 years after onset. Approximately 4 million Americans have Alzheimer's disease. One in 10 persons over 65, and nearly half of those over 85, have Alzheimer's disease. Alzheimer's disease costs U.S. society at least $100 billion a year, with neither Medicare nor most private health insurances covering the long-term care most patients need.

Alzheimer's disease is characterized by the progressive accumulation of the amyloid-beta (Aβ) protein in limbic and association cortices, where some of it precipitates to form a range of amorphous and compacted (fibrillar) extracellular plaques. These plaques, particularly the more compacted ones, are associated with dystrophic neurites (altered axons and dendrites), activated microglia and reactive astrocytes. Cleavage of the amyloid precursor protein (APP) by the β and γ-secretases releases both the Aβ1-40 and Aβ1-42 peptides, the latter being more prone to aggregation and induction of neurotoxicity.

Although Alzheimer's disease is associated with local innate immune responses, the induction of systemic adaptive immune responses to Aβ in mouse models of Alzheimer's disease has been found to be beneficial for both the neuropathological and behavioral changes that these mice develop. Beneficial response to a single Aβ immunization includes enhanced clearance of Aβ accumulation in the hippocampus of the mice immunized.

A human clinical trial in which an Aβ1-42 synthetic peptide was administered parenterally with the adjuvant QS21 to patients with Alzheimer's disease was discontinued when approximately 5% of 300 treated patients developed what appeared to be a self-limited aseptic meningoencephalitis. One hypothetical cause of this reaction was an immune response to Aβ. Therefore, any therapeutic value of Aβ peptide administration to Alzheimer's disease patients was curtailed due to the life threatening response of just a small percentage of the Alzheimer patient population to whom administration of Aβ1-42 synthetic peptides appeared to be an incorrect therapeutic choice.

### SUMMARY OF THE INVENTION

This invention provides, in one embodiment, an isolated polypeptide, wherein said polypeptide consists of an amino acid sequence as set forth in SEQ ID NOs: 1, 3-6, 9, 12, 22, or 23.

This invention further provides, in one embodiment, a composition comprising an isolated polypeptide of this invention.

This invention further provides, in one embodiment, a method of optimized treatment of a subject afflicted with a disease or disorder associated with amyloid beta accumulation, comprising determining expression of a particular HLA-DRB1 allele in a sample derived from a subject and administering an optimized peptide to the subject whereby, administration of the optimized peptide to the subject expressing a particular HLA-DRB1 allele results in an optimal treatment for the subject.

This invention further provides, in one embodiment, a method of optimized immunization against a disease characterized by amyloid beta accumulation in a subject, comprising determining expression of a particular HLA-DDRB1 allele in a sample derived from a subject and administering an optimized peptide to the subject, whereby administration of the optimized peptide to the subject expressing a particular HLA-DRB1 allele reduces a risk of disease characterized by amyloid beta accumulation or reduces severity of disease characterized by amyloid beta accumulation for the subject.

According to this aspect, and in on embodiment, the peptide is cultured ex vivo or in vitro with an antigen presenting cell, a T cell or a combination thereof and said antigen presenting cell cell, T cell or combination thereof is then administered to said subject.

In some embodiments, this invention provides for the use of a peptide having a an amino acid sequence as set forth in any one of SEQ ID NOs: 3-7, or any combination thereof for the preparation of a medicament for use in treating a subject afflicted with a disease or disorder associated with amyloid beta accumulation, reduces a risk of disease characterized by amyloid beta accumulation or reducing the severity of disease characterized by amyloid beta accumulation for said subject wherein said subject expresses an HLA-DRB1 0301 allele. In some embodiments, this invention provides for the use of a peptide having a an amino acid sequence as set forth in any one of SEQ ID NOs: 1, 2, 7, 14, 20, 21, and 27-34 or any combination thereof for the preparation of a medicament for use in treating a subject afflicted with a disease or disorder associated with amyloid beta accumulation, or reducing the severity of disease characterized by amyloid beta accumulation for said subject, wherein said subject expresses an HLA-DRB1 1501 allele,

In some embodiments, this invention provides for the use of a peptide having a an amino acid sequence as set forth in any one of SEQ ID NOs: 3, 4, 5, 7, 8, 22-28, or any combination thereof or any combination thereof for the preparation of a medicament for use in treating a subject afflicted with a disease or disorder associated with amyloid beta accumulation, or reducing the severity of disease characterized by amyloid beta accumulation for said subject, wherein said subject expresses an HLA-DRB1 0101.

In some embodiments, this invention provides for the use of a peptide having a an amino acid sequence as set forth in any one of SEQ ID NOs: 7, 9, 10, 24-26 or any combination thereof or any combination thereof for the preparation of a medicament for use in treating a subject afflicted with a disease or disorder associated with amyloid beta accumulation, or reducing the severity of disease characterized by amyloid beta accumulation for said subject, wherein said subject expresses an HLA-DRB1 1301 allele.

In some embodiments, this invention provides for the use of a peptide having a an amino acid sequence as set forth in any one of SEQ ID NOs: 3-5, 7, or any combination thereof or any combination thereof for the preparation of a medicament for use in treating a subject afflicted with a disease or disorder associated with amyloid beta accumulation, or reducing the severity of disease characterized by amyloid beta accumulation for said subject, wherein said subject expresses an 1502 allele.

In some embodiments, this invention provides for the use of a peptide having a an amino acid sequence as set forth in any one of SEQ ID NOs: 7, 9, 10,11, 19, 24-26, or any combination thereof for the preparation of a medicament for use in treating a subject afflicted with a disease or disorder associated with amyloid beta accumulation, or reducing the severity of disease characterized by amyloid beta accumulation for said subject, wherein said subject expresses an HLA-DRB1 0404 allele.

In some embodiments, this invention provides for the use of a peptide having a an amino acid sequence as set forth in any one of SEQ ID NOs: 7, 12,13, 22-24, or any combination thereof for the preparation of a medicament for use in treating a subject afflicted with a disease or disorder associated with amyloid beta accumulation, or reducing the severity of disease characterized by amyloid beta accumulation for said subject, wherein said subject expresses an HLA-DRB1 1001 allele.

In some embodiments, this invention provides for the use of a peptide having a an amino acid sequence as set forth in any one of SEQ ID NOs: 7, 9, 11, 13, 15, 18, 22-24, or any combination thereof for the preparation of a medicament for use in treating a subject afflicted with a disease or disorder associated with amyloid beta accumulation, or reducing the severity of disease characterized by amyloid beta accumulation for said subject, wherein said subject expresses an HLA-DRB1 0401 allele.

According to this aspect, and in some embodiments, the medicament further comprises a neuroprotective compound or a neurotrophic factor.

According to this aspect, and in some embodiments, the peptide is cultured ex vivo or in vitro with an antigen presenting cell, a T cell or a combination thereof and said dendritic cell, T cell or combination thereof is then used for the preparation of said medicament.

This invention provides, in one embodiment, a method of determining responsiveness of a subject afflicted with a disease or disorder associated with amyloid beta accumulation to an amyloid beta peptide-dependent treatment regimen, comprising determining expression of a particular HLA-DRB1 allele in a sample derived from a subject, wherein if the subject expresses an HLA-DRB1 1501 allele, an HLA-DRB1 0301, an HLA-DRB1 0101 allele, an HLA-DRB1 1301 allele, an HLA-DRB1 1502 allele, an HLA-DRB1 0404 allele, an HLA-DRB1 1001 allele, an HLA-DRB1 0401 allele, an HLA-DRB1 1104 allele, an HLA-DRB1 0402 allele, an HLA-DRB1 04011 allele, an HLA-DRB1 1302 allele, an HLA-DBR1 0403 allele, a HLA-DRB1 1102 allele, an HLA-DRB1 0103 allele, an HLA-DRB1 0407 allele, an HLA-DRB1 0302 allele, or an HLA-DRB1 1404 allele, then the subject will be responsive to an amyloid beta peptide-dependent regimen.

This invention further provides, in one embodiment, a method of optimized treatment of a Caucasian subject afflicted with a disease or disorder associated with amyloid beta accumulation, comprising administering at least one polypeptide to the Caucasian subject, wherein the polypeptide has an amino acid sequence as set forth in any of SEQ ID NOs: 1-7, 14, 20-34, or a combination thereof.

This invention further provides, in one embodiment, a method of optimized treatment of an Asian or Arab subject afflicted with a disease or disorder associated with amyloid beta accumulation, comprising administering at least one polypeptide to the Asian or Arab subject, wherein the polypeptide has an amino acid sequence as set forth in any of SEQ ID NOs: 1-7, 9, 11-15, 18, 20-24, 27-34, or a combination thereof.

This invention further provides, in one embodiment, a method of optimized treatment of an African subject afflicted with a disease or disorder associated with amyloid beta accumulation, comprising administering at least one polypeptide to the African subject, wherein the polypeptide has an amino acid sequence as set forth in SEQ ID NOs: 3-7, 9, 10, 12, 13, 22, 23, or 24, or a combination thereof.

This invention further provides, in one embodiment, a method of optimized treatment of an African American subject afflicted with a disease or disorder associated with amyloid beta accumulation, comprising administering at least one polypeptide to the African American subject, wherein the polypeptide has an amino acid sequence as set forth in SEQ ID NOs: 3-7, 12, 13, 22, 23, or 24, or a combination thereof.

This invention provides, in one embodiment, a method for assessing responsiveness of a subject to an amyloid beta peptide-dependent treatment regimen, comprising, determining expression of a particular HLA-DRB1 allele in a sample derived from a subject, wherein if the subject expresses any two of the following alleles, an HLA-DRB1 1101 allele, an HLA-DRB1 0801 allele, an HLA-DRB1 0102 allele, or an HLA-DRB1 00170 allele then the subject is considered to be non-responsive to an amyloid beta peptide-dependent treatment regimen.

### BRIEF DESCRIPTION OF THE FIGURES

FIG. 1 illustrates the epitope specificity and DRB1 allele restriction of Aβ-reactive T-cell lines. Aβ-reactive T-cell lines were generated from the PBMCs of two human subjects bearing the DRB1 allele 1501/1502 (FIG. 1A, 1C, 1E) or 1301/0401 (FIG. 1B, 1D, 1F), as described in Materials and Methods. Aβ-reactive T-cell epitope specificity was determined by stimulation of the T-cell lines with irradiated autologous PBMCs in the presence of increasing concentrations of Aβ peptides, and T-cell proliferation was measured 72 hours later. (A, B) T-cell line proliferation induced by Aβ1-42 and the two overlapping peptides Aβ1-28 and Aβ15-42. For T-cell line 1, the SI values obtained at 5 μg peptide/ml were 21.1, 2.9, and 15.8 for Aβ1-42, Aβ1-28, and Aβ15-42, respectively; for T-cell line 2, the corresponding SI values were 8.3, 0.7, and 7.8, respectively. (C, D) T-cell line proliferation induced by nested peptides of the Aβ15-42 region. For T-cell line 1, the SI values obtained at 5 μg peptide/ml were 10.2, 17.3, and 12.6 for Aβ22-36, Aβ25-39, and Aβ28-42, respectively; for T-cell line 2, the SI values obtained at 5 μg peptide/ml were 6.4, 6.2, and 6.1 for Aβ16-30, Aβ17-31, and Aβ18-32, respectively. (E, F) The DRB1 alleles predominantly presenting the Aβ peptides were identified by stimulation of the T-cell lines with Aβ1-42 in the presence of irradiated autologous or semi-autologous PBMCs. The results shown are from one representative experiment out of three performed for T-cell lines 1 and 2, respectively.

FIG. 2 illustrates the T-cell-epitope analysis of Aβ-immunized DR15 and DR4 humanized mice. Humanized mice bearing the HLA-DR15 and HLA-DR4 alleles were immunized by subcutaneous injection of Aβ1-42 emulsified in CFA and a subsequent booster injection of Aβ1-42 emulsified in IFA. On day 18, the mice were killed and their splenocyte-derived T cells were stimulated in the presence of increasing concentrations of Aβ peptides. T-cell proliferation was measured 72 hours later, as described in Materials and Methods. (A, B) T-cell proliferation induced by Aβ1-42 and the two overlapping peptides Aβ1-28 and Aβ15-42. For DR15 mice, SI values obtained at 10 μg/ml were 10.8, 4.5, and 14.2 for Aβ1-42, Aβ1-28, and Aβ15-42, respectively. For DR4 mice, the corresponding SI values were 8.4, 1.2, and 3.7 for Aβ1-42, Aβ1-28, and Aβ15-42, respectively. (C, D) T-cell proliferation induced by nested peptides of the Aβ15-42 region. For DR15 mice, the SI values obtained at 10 μg/ml were 8.8 for Aβ25-39 and 12.2 for Aβ28-42. For DR4 mice, the SI values obtained at 10 μg/ml were 5.9, 6.1, and 9.2 for Aβ16-30, Aβ17-31, and Aβ18-32, respectively. (E, F) T-cell proliferation in the presence of Aβ1-42 and HLA-DR-blocking antibodies. The results shown in each case are the values obtained for four mice (means±SD) in one representative experiment out of three performed.

FIG. 3 illustrates that Anti-IgG1 is the predominant Aβ1-15-specific antibody induced in DR15 Tg mice. HLA-DR15 humanized mice (n=4) were immunized by subcutaneous injection of Aβ1-42 emulsified in CFA and given two subsequent booster injections of Aβ1-42 emulsified in IFA at 2-week intervals. (A) Different Aβ-specific (left panel) and OVA-specific (right panel) antibody isotypes produced in serum samples 2 weeks after the last immunization were measured by ELISA, as described in Materials and Methods. Antibody titers obtained in each mouse are shown. (B) Epitope specificities of the Aβ antibodies produced in DR15 Tg and B6SJLF1 control mice were identified by ELISA for anti-Aβ1-42, anti-Aβ1-15, and anti-Aβ15-42 antibodies, as described in Materials and Methods. Columns represent the amounts of IgG in each group (means±SD).

FIG. 4 demonstrates the specific immune response to Aβ after long-term immunization of APP/DR15 Tg mice. APP/DR15 Tg mice (n=3) aged 7 months were immunized with Aβ emulsified in CFA and injected intravenously with PTX. Mice were then given two subsequent booster injections of Aβ42 in IFA at 3.5-week intervals. The mice were killed 3.5 weeks after the last injection (aged 9.5 months). Their splenocytes were analyzed for Aβ-induced T-cell proliferation and cytokine production. (A) T-cell proliferation was measured by 3[H] thymidine incorporation. The cytokines IL-2 and IFN-γ were measured by ELISA 24 hours (B) and 48 hours (C) after Aβ stimulation, respectively. The results shown in each case are the values obtained for three mice (means±SD) in one representative experiment out of two performed.

FIG. 5 demonstrates that prophylactic Aβ immunization of APP/HLA-DR15 Tg mice enhances Aβ clearance from their brains. APP/DR15 Tg mice aged 7 months were immunized with Aβ emulsified in CFA and injected intravenously with PTX (n=3) or with Aβ emulsified in IFA (n=4), and were given two subsequent booster injections of Aβ1-42 in IFA at 3.5-week intervals. Control mice (n=5) were similarly immunized with adjuvant alone. At 9.5 months of age, the mice were killed and their brains were excised and analyzed for Aβ by immunohistochemistry, as described in Materials and Methods. Brain sections were immunolabeled with anti-Aβ (green) and counterstained with TO-PRO 3 (blue). (A) Representative sections from micevaccinated with Aβ and with adjuvant only are shown. (B) The area occupied by Aβ on each 12-μm-thick section was quantified using the Volocity 3S Image Analysis software, as described in Materials and Methods. Columns represent the Aβ area per brain section in each group of mice (means±SD). (C) Response of Aβ-antibody isotypes in serum samples of vaccinated mice, quantified by ELISA. Columns represent the amounts of IgG in each group (means±SD).

FIG. 6 demonstrates that microglial activation is reduced in APP/DR15 Tg mice immunized prophylactically with Aβ. Brain sections obtained from a representative adjuvant-vaccinated (control; upper panels) and Aβ/IFA-vaccinated mouse (lower panels) were immunolabeled with anti-CD11b, anti-Aβ and anti-CD4, and counterstained with TO-PRO 3 (blue), as described in Materials and Methods. (A) Three-dimensional representations of Z-stalk images taken from the hippocampi of representative sections show separate panels of CD11b (green) and Aβ (red) immunolabeling and their merged appearance. Arrows in the “merge” panels point to co-localized activated microglia and Aβ plaques. CD4 immunolabeling (green) of representative sections are shown in the right panels. (B) The area occupied by CD11b+ cells (left panel) and their fluorescence intensity on each 12-μm-thick section was quantified using the Volocity 3S Image Analysis software. Columns represent the CD11b area or intensity per brain section in each group of mice (means±SD).

### DETAILED DESCRIPTION OF THE PRESENT INVENTION

This invention provides, inter alia, novel polypeptides, compositions, and methods of use thereof for optimized treatment of diseases or disorders associated with amyloid beta protein (Aβ) accumulation in a subject and methods for optimized immunization against diseases characterized by Aβ accumulation in a subject

Various methods for personalizing treatment, prevention, or reduction of the incidence or severity of Alzheimer's disease and other disorders related to the accumulation of extracellular Aβ protein-containing plaques (e.g., an amyloid fibril disorder) are described herein, representing embodiments of the invention. An obstacle to a method of treatment administering an Aβ1-42 peptide to a subject in need is that not all individuals afflicted with a disease or disorder associated with Aβ accumulation respond favorably to this therapy. The response of an individual's T cell to different epitopes present within the full-length Aβ1-42 polypeptide varies widely. For example, the response of an individual's T cells to Aβ may vary depending on the MHC Class II alleles the individual carries.

Accordingly, methods of the instant invention provide for the identification of an optimized treatment regime that will most effectively meet the needs of an individual subject.

A personalized medicine approach seeks to identify whether a given individual will respond favorable to a given treatment or intervention prior to administering it, rather than relying on “standards” representing an average person in a group or population. Therefore, optimizing selection of an Aβ peptide for a subject afflicted with a disease or disorder associated with Aβ accumulation may be particularly useful for optimizing treatment of such a subject.

The methods of this invention include administering to a subject an optimized Aβ peptide and/or an optimized composition that elicits a beneficial T cell response; for example, a T cell response reducing Aβ accumulation in the brain of a subject, without causing an encephalitic response.

In one embodiment, the present invention is directed to polypeptides, which include polypeptides which are derived from an Aβ polypeptide, and which peptides, in some embodiments are capable of eliciting a T cell response upon administration to a subject.

In one embodiment, the phrase “T cell response” refers to T cell proliferation, T cell stimulation or activation, T cell elaboration of a cytokine, T cell responsiveness to a cytokine, T cell effector function, reduction of Aβ accumulation in a subject administered an Aβ polypeptide, or combinations thereof.

In one embodiment, the Aβ polypeptide carries an epitope which produces a positive T cell proliferation response in a subject carrying a particular HLA-DRB1 allele. The T-cell response can be determined in a variety of ways, including by exposing the peptide to peripheral blood monocyte cells (PBMC). In some embodiments, the T cell response is in the central nervous system, for example in the brain.

This invention provides, in some embodiments, polypeptides for use in stimulating specific immune responses useful in treating, suppressing, inhibiting, or abrogating a disease associated with amyloid beta accumulation in a subject.

Table 1, hereinbelow provides polypeptides and/or polypeptides for use in the compositions and methods of this invention.

In one embodiment, a polypeptide of this invention has an amino acid sequence, which corresponds to or is homologous to SEQ ID NOs: 1-34 as shown in Table 1. In one embodiment, a polypeptide of this invention has an amino acid sequence, which corresponds to or is homologous to SEQ ID NOs: 1, 3-6, 9, 12, 22 or 23 as shown in Table 1.

TABLE 1
SEQ ID NOs: 1-34

| SEQ ID NO | Aβ peptide | Amino acid sequence |
|---|---|---|
| 1 | 28-37 | KGAIIGLMVG |
| 2 | 25-39 | GSNKGAIIGLMVGGV |
| 3 | 18-29 | VFFAEDVGSNKG |
| 4 | 21-32 | AEDVGSNKGAII |
| 5 | 24-35 | VGSNKGAIIGLM |
| 6 | 27-38 | NKGAIIGLMVGG |
| 7 | 15-42 | QKLVFFAEDVGSNKGAIIGLMVGGVVIA |
| 8 | 15-34 | QKLVFFAEDVGSNKGAIIGL |
| 9 | 21-30 | AEDVGSNKGA |
| 10 | 17-31 | LVFFAEDVGSNKGAI |
| 11 | 18-32 | VFFAEDVGSNKGAII |
| 12 | 18-27 | VFFAEDVGSN |
| 13 | 15-29 | QKLVFFAEDVGSNKG |
| 14 | 21-35 | AEDVGSNKGAIIGLM |
| 15 | 16-30 | KLVFFAEDVGSNKGA |
| 16 | 1-42 | DAEFRHDSGYEVHHQKLVFFAEDVGSNKGAIIGLMVGGVVIA |
| 17 | 1-14 | DAEFREDSGYEVHH |
| 18 | 16-32 | KLVFFAEDVGSNKGAII |
| 19 | 17-32 | LVFFAEDVGSNKGAII |
| 20 | 22-36 | EDVGSNKGAIIGLMV |
| 21 | 28-42 | KGAIIGLMVGGVVIA |
| 22 | 15-26 | QKLVFFAEDVGS |
| 23 | 16-27 | KLVFFAEDVGSN |
| 24 | 17-28 | LVFFAEDVGSNK |
| 25 | 19-30 | FFAEDVGSNKGA |
| 26 | 20-31 | FAEDVGSNKGAI |
| 27 | 22-33 | EDVGSNKGAIIG |
| 28 | 23-34 | DVGSNKGAIIGL |
| 29 | 25-36 | GSNKGAIIGLMV |
| 30 | 26-37 | SNKGAIIGLMVG |
| 31 | 28-39 | KGAIIGLMVGGV |
| 32 | 29-40 | GAIIGLMVGGVV |
| 33 | 30-41 | AIIGLMVGGVVI |
| 34 | 31-42 | IIGLMVGGVVIA |

In one embodiment, the polypeptides of this invention include any polypeptide as herein described or any polypeptide which is homologous thereto, which produces a positive T cell proliferation response in a subject carrying a particular HLA-DRB1 allele.

The terms “peptides”, “proteins”, and “polypeptides” are used interchangeably herein.

In some embodiments, the term “peptide” refers to native peptides (either degradation products, synthetically synthesized peptides or recombinant peptides) and peptidomimetics (typically, synthetically synthesized peptides), such as peptoids and semipeptoids which are peptide analogs, which may have, for example, modifications rendering the peptides more stable while in a body or more capable of penetrating into bacterial cells. Such modifications include, but are not limited to N terminus modification, C terminus modification, peptide bond modification, including, but not limited to, CH2-NH, CH2-S, CH2-S═O, O═C—NH, CH2-O, CH2-CH2, S═C—NH, CH═CH or CF═CH, backbone modifications, and residue modification. Methods for preparing peptidomimetic compounds are well known in the art and are specified, for example, in Quantitative Drug Design, C. A. Ramsden Gd., Chapter 17.2, F. Choplin Pergamon Press (1992), which is incorporated by reference as if fully set forth herein. Further details in this respect are provided hereinunder.

Peptide bonds (—CO—NH—) within the peptide may be substituted, for example, by N-methylated bonds (—N(CH3)-CO—), ester bonds (—C(R)H—C—O—O—C(R)—N—), ketomethylen bonds (—CO—CH2-), *-aza bonds (—NH—N(R)—CO—), wherein R is any alkyl, e.g., methyl, carba bonds (—CH2-NH—), hydroxyethylene bonds (—CH(OH)—CH2-), thioamide bonds (—CS—NH—), olefinic double bonds (—CH═CH—), retro amide bonds (—NH—CO—), peptide derivatives (—N(R)—CH2-CO—), wherein R is the “normal” side chain, naturally presented on the carbon atom.

These modifications can occur at any of the bonds along the peptide chain and even at several (2-3) at the same time.

Natural aromatic amino acids, Trp, Tyr and Phe, may be substituted for synthetic non-natural acid such as TIC, naphthylelanine (Nol), ring-methylated derivatives of Phe, halogenated derivatives of Phe or o-methyl-Tyr.

In addition to the above, the peptides of the present invention may also include one or more modified amino acids or one or more non-amino acid monomers (e.g. fatty acids, complex carbohydrates etc).

As used herein in the specification and in the claims section below the term “amino acid” or “amino acids” is understood to include the 20 naturally occurring amino acids; those amino acids often modified post-translationally in vivo, including, for example, hydroxyproline, phosphoserine and phosphothreonine; and other unusual amino acids including, but not limited to, 2-aminoadipic acid, hydroxylysine, isodesmosine, nor-valine, nor-leucine and ornithine. Furthermore, the term “amino acid” includes both D- and L-amino acids.

In one embodiment, an isolated polypeptide or a polypeptide for use in the compositions and methods of this invention comprises a peptide of contiguous amino acids selected from amino acids 1-42 of SEQ ID No. 16. In one embodiment, an isolated polypeptide comprises contiguous amino acids from amino acid 1-14 of SEQ ID No. 16. In one embodiment, an isolated polypeptide comprises contiguous amino acids from amino acid 15-42 of SEQ ID No. 16. In one embodiment, an isolated polypeptide of this invention comprises at least 8 contiguous amino acids from amino acid 15-42 of SEQ ID No. 16. In one embodiment, an isolated polypeptide of this invention comprises at least 10 contiguous amino acids from amino acid 15-42 of SEQ ID No. 16. In one embodiment, an isolated polypeptide of this invention comprises at least 15 contiguous amino acids from amino acid 15-42 of SEQ ID No. 16. In one embodiment, an isolated polypeptide of this invention comprises at least 6 contiguous amino acids from amino acid 1-14 of SEQ ID No. 16 and at least 6 contiguous amino acids from amino acid 15-42 of SEQ ID No. 16.

The polypeptides of this invention can be produced by any synthetic or recombinant process such as is well known in the art. Polypeptides can further be modified to alter biophysical or biological properties by means of techniques known in the art. For example, the polypeptide can be modified to increase its stability against proteases, or to modify its lipophilicity, solubility, or binding affinity to its native receptor.

In one embodiment, the invention provides a composition including at least one polypeptide as described herein.

In one embodiment, a polypeptide of this invention carries an epitope for a T cell proliferation response. Table 2 in Example 5 provides an exemplary list of peptides carrying an epitope for T cell proliferation response with regards to specific HLA-DRB1 alleles.

In one embodiment, the polypeptide of interest comprises an epitope whose presentation specifically on an MHC class II HLA-DRB1 allele is desired.

In one embodiment, the term “epitope” refers to a fragment of an antigen, which is specifically recognized by a part of the immune response repertoire. This invention provides, in some embodiments, polypeptides, whose sequence corresponds to an epitope specifically recognized by an immune response mediator, for example, and in some embodiments a T cell response, and in other embodiments, a B cell response, and in other embodiments, both B and T cell responses.

The polypeptides of this invention may comprise a longer sequence of amino acids, than that which constitutes the epitope. In some embodiments, a polypeptide of this invention may contain one or more distinct epitopes. An epitope may refer, in some embodiments, to an immunogenic portion of a multichain polypeptide, i.e., which is encoded by distinct open reading frames. The terms epitope, peptide, and polypeptide all refer to a series of amino acids connected one to the other by peptide bonds between the alpha-amino and alpha-carboxy groups of adjacent amino acids, and may contain or be free of modifications such as glycosylation, side chain oxidation, or phosphorylation, provided such modifications, or lack thereof, do not destroy immunogenicity.

In some embodiments, the epitope (peptide, polypeptide, antigen) is as small as possible while still maintaining immunogenicity. Immunogenicity is indicated by the ability to elicit an immune response, as described herein, for example, by the ability to bind an MHC class II HLA-DRB1 molecule and to induce a T cell response, e.g., by measuring T cell cytokine production.

In some embodiments, the terms “antigen” or “immunogen” refer to a peptide, protein, polypeptide which is immunogenic, that is capable of eliciting an immune response in a mammal, and therefore contains at least one and may contain multiple epitopes.

In one embodiment, an isolated polypeptide or polypeptide for use in the compositions and methods of this invention comprises an optimized peptide, capable of producing an immune response. In one embodiment, a polypeptide comprises a T cell epitope. In one embodiment, a polypeptide comprises a B cell epitope. In one embodiment, a polypeptide comprises a T cell epitope and a B cell epitope

In one embodiment, a composition used in the methods of this invention comprises a polypeptide comprising a T cell epitope and a polypeptide comprising a B cell epitope.

In one embodiment “immune response” refers to the development of a humoral (antibody mediated) or a cellular (mediated by antigen-specific T cells or their secretion products) response directed against an antigen in a vertebrate individual. Such a response can be an active response induced by administration of immunogen or a passive response induced by administration of antibody or primed T-cells or B cells which can act as antigen presenting cells. A cellular immune response is elicited by the presentation of polypeptide epitopes in association with Class I or Class II MEC molecules to activate antigen-specific CD4⁺T helper cells and/or CD8⁺ cytotoxic T cells. The response may also involve activation of monocytes, macrophages, NK cells, basophils, dendritic cells, astrocytes, microglia cells, eosinophils or other components of innate immunity. The presence of a cell-mediated immunological response can be determined by standard proliferation assays (CD4⁺ T cells) or CTL (cytotoxic T lymphocyte) assays known in the art.

In one embodiment of this invention, a “B cell epitope” refers to the amino acid residues in an antigen that result in specificity of binding to a B cell receptor. In one embodiment of this invention, a “T cell epitope” refers to the amino acid residues in an antigen that result in specificity of binding to a T cell receptor.

This invention is directed, in some embodiments, to methods of use which depend upon specific responses elicited as a consequence of exposure to a polypeptide of this invention and such responses may involve both components of the immune system: the humoral component and the cellular component. Thus, “immune response” in the context of the invention includes any component of the humoral or cellular immune response.

The responses elicited as part of the methods of this invention, or as a consequence of exposure to a polypeptide of this invention may comprise elaboration of cytokines and other signals necessary to give rise to soluble antibody production, immune cell activation, effector function, and others, as will be appreciated by the skilled artisan. Each and all of these markers of immune responses are measurable by known methods, as will be appreciated by the skilled artisan. Such methods are well known in the art, see for example “Current Protocols in Immunology” Coligan, J. E., ed. (2007).

As will be understood by those of skill in the art, a single polypeptide of this invention may contain both T and B cell epitopes, such that modification of both may alter both the humoral and cellular arms of the immune system, and comprise envisioned methods and/or compositions/polypeptides of this invention.

In another embodiment, the present invention comprises compositions comprising optimized peptides as herein described, capable of producing a positive T cell proliferation response in a subject carrying a particular HLA-DRB1 allele.

In one embodiment, HLA-DRB1 allele refers to alleles of the major histocompatibility complex, MHC class II, DR beta 1 gene. HLA-DRB1 encodes the most prevalent subunit of the HLA-DR cell surface receptor.

In one embodiment, the present invention comprises methods of use of an optimized polypeptide as herein described or compositions comprising the same in optimizing treatment of a subject afflicted with a disease or disorder associated with Aβ accumulation, by administration the optimized polypeptide or composition to the subject. In another embodiment, the present invention comprises methods of use of an optimized polypeptide as herein described or compositions comprising the same in optimizing immunization of a subject against a disease characterized by Aβ accumulation, by administration the optimized polypeptide or composition to the subject.

In one embodiment, the compositions of the invention comprise an agent, which is neuroprotective, or in another embodiment, the compositions of the invention comprise an agent, which is a neurotrophic factor.

In some embodiments, the neuorprotective agent is a cytokine, which in some embodiments, is interferon-γ, interleukin 4 (IL-4), IL10, IL17, or TGFβ.

In some embodiments, the neurotrophic factor is nerve growth factor (NGF), insulin growth factor (IGF), brain-derived neurotrophic factor. (BDNF) or ciliary neurotrophic factor (CNTF).

In one embodiment, the compositions of the invention comprise an agent which increases levels of interferon-γ.

In one embodiment, the term “agent” refers to any molecule which satisfies the indicated purpose. In some embodiments, “agent” is a nucleic acid, an oligonucleotide, an oligopeptide, a polypeptide, a protein, a functional fragment thereof, a small molecule, or any chemical moiety suitable for the indicated purpose.

In one embodiment, the agent is a nucleic acid. In some embodiments, the term “nucleic acid” refers to polynucleotide or to oligonucleotides such as deoxyribonucleic acid (DNA), and, where appropriate, ribonucleic acid (RNA) or mimetic thereof. The term should also be understood to include, as equivalents, analogs of either RNA or DNA made from nucleotide analogs, and, as applicable to the embodiment being described, single (sense or antisense) and double-stranded polynucleotides. This term includes oligonucleotides composed of naturally occurring nucleobases, sugars and covalent internucleoside (backbone) linkages as well as oligonucleotides having non-naturally-occurring portions, which function similarly. Such modified or substituted oligonucleotides are often preferred over native forms because of desirable properties such as, for example, enhanced cellular uptake, enhanced affinity for nucleic acid target and increased stability in the presence of nucleases.

As will be appreciated by one skilled in the art, a fragment or derivative of a nucleic acid sequence or gene that encodes for a protein or peptide can still function in the same manner as the entire, wild type gene or sequence. Likewise, forms of nucleic acid sequences can have variations as compared to wild-type sequences, nevertheless encoding the protein or peptide of interest, or fragments thereof, retaining wild-type function exhibiting the same biological effect, despite these variations. Each of these represents a separate embodiment of this present invention.

The nucleic acids of this invention can be produced by any synthetic or recombinant process such as is well known in the art. Nucleic acids can further be modified to alter biophysical or biological properties by means of techniques known in the art. For example, the nucleic acid can be modified to increase its stability against nucleases (e.g., “end-capping”), or to modify its lipophilicity, solubility, or binding affinity to complementary sequences.

DNA according to the invention can also be chemically synthesized by methods known in the art. For example, the DNA can be synthesized chemically from the four nucleotides in whole or in part by methods known in the art. DNA can also be synthesized by preparing overlapping double-stranded oligonucleotides, filling in the gaps, and ligating the ends together, by standard methods known in the art. DNA expressing functional homologues of the protein can be prepared from wild-type DNA by site-directed mutagenesis. The DNA obtained can be amplified by methods known in the art. One suitable method is the polymerase chain reaction (PCR) method. Such methods are well known in the art, see for example, U.S. Pat. No.4,683,195, “Current Protocols in Molecular Biology” Volumes I-III Ausubel, R. M., ed. (1994); Ausubel et al., “Current Protocols in Molecular Biology”, John Wiley and Sons, Baltimore, Md. (1989); Perbal, “A Practical Guide to Molecular Cloning”, John Wiley & Sons, New York (1988); Watson et al., “Recombinant DNA”, Scientific American Books, New York; Birren et al. (eds) “Genome Analysis: A Laboratory Manual Series”, Vols. 1-4, Cold Spring Harbor Laboratory Press, New York (1998); methodologies as set forth in U.S. Pat. Nos. 4,666,828; 4,683,202; 4,801,531; 5,192,659 and 5,272,057; U.S. Pat. Nos. 3,791,932; 3,839,153; 3,850,752; 3,850,578; 3,853,987; 3,867,517; 3,879,262; 3,901,654; 3,935,074; 3,984,533; 3,996,345; 4,034,074; 4,098,876; 4,879,219; 5,011,771 and 5,281,521; “Oligonucleotide Synthesis” Gait, M. J., ed. (1984); “Nucleic Acid Hybridization” Hames, B. D., and Higgins S. J., eds. (1985); “Transcription and Translation” Hames, B. D., and Higgins S. J., eds. (1984); “Animal Cell Culture” Freshney, R. I., ed. (1986); “Immobilized Cells and Enzymes” IRL Press, (1986); “A Practical Guide to Molecular Cloning” Perbal, B., (1984) and “Methods in Enzymology” Vol. 1-317, Academic Press; “PCR Protocols: A Guide To Methods And Applications”, Academic Press, San Diego, Calif. (1990); Marshak et al., “Strategies for Protein Purification and Characterization—A Laboratory Course Manual” CSHL Press (1996); all of which are incorporated by reference as if fully set forth herein. Other general references are provided throughout this document. The procedures therein are well known in the art and are provided for the convenience of the reader. All the information contained in any reference cited herein is to be understood to be incorporated by reference in its entirety.

Methods for modifying nucleic acids to achieve specific purposes are disclosed in the art, for example, in Sambrook et al. (1989). Moreover, the nucleic acid sequences of the invention can include one or more portions of nucleotide sequence that are non-coding for the protein of interest. Variations in the DNA sequences, which are caused by point mutations or by induced modifications (including insertion, deletion, and substitution) to enhance the activity, half-life or production of the polypeptides encoded thereby, are also encompassed in the invention.

In some embodiments, the agent, which increases levels of interferon-γ is a nucleic acid encoding the protein, or a functional fragment thereof. In some embodiments, the agent is the protein or a functional polypeptide fragment thereof. In some embodiments, the term “functional fragment” refers to the ability of the fragment to effect the indicated purpose of the cited agent.

In some embodiments, the nucleic acid agent, which increases levels of interferon-γ, encodes the protein. In some embodiments, the nucleic acid may have a sequence corresponding to or homologous to any known sequence encoding the protein, or one inducing the same. For example, and in some embodiments, the nucleic acid may have a sequence corresponding to or homologous to that set forth in NCBI's Genbank Accession No.: NM000619, EF173872, E12009, E11745, E15793, E15653, E06017, E00832, E00692, E00663, E00611, E00388, E00380, E00270, E00228, E00226, E00180, E00119, E00118, and others. In some embodiments, the nucleic acid agent, which increases brain levels of interferon-γ encodes interleukin-2 (IL-2), IL-12, IL-18, interferon α, interferon β or TNF α, comprising any sequence known to encode the same, for example, the nucleic acid may have a sequence corresponding to or homologous to that set forth in NCBI's Genbank Accession No.: NM_{—}000594, NM_{—}000882, NM_{—}002176, or others, as will be appreciated by one skilled in the art.

In some embodiments, the agent that increases levels of interferon-γ comprises the protein itself or a functional fragment or derivative thereof. In some embodiments, the polypeptide may have a sequence corresponding to or homologous to any known sequence for the protein, or one inducing the same. For example, and in some embodiments, the polypeptide may have an amino acid sequence corresponding to or homologous to that set forth in NCBI's Genbank Accession No.: AAM28885, CAA44325, CAP17327, AAK95388, ABM53145, AAP20100, AAP20098, AAK53058, CAA00226, AAA53230, AAA16521, CAA44328, CAA44329, CAA44330, CAA44326, and others. In some embodiments, the polypeptide agent, which increases brain levels of interferon-γ is interleukin-2 (IL-2), IL-12, IL-18, interferon α, interferon β or TNF α, comprising any sequence known corresponding to the same, for example, the polypeptide may have an amino acid sequence corresponding to or homologous to that set forth in NCBI's Genbank Accession No.: AAA52716, AAA52724, AAA52713, AAC41702, AAO26357, NP_{—}002167, CAA01199, AAA59140, AAA98792, AAD16432, ABM53138, NP_{—}001553, AAK95950, CAC01436 or others, as will be appreciated by one skilled in the art.

Polypeptide homology for any polypeptide sequence listed herein may be determined by immunoblot analysis, or via computer algorithm analysis of amino acid sequences, utilizing any of a number of software packages available, via methods well known to one skilled in the art. Some of these packages may include the FASTA, BLAST, MPsrch or Scanps packages, and may employ the use of the Smith and Waterman algorithms, and/or global/local or BLOCKS alignments for analysis, for example.

Homology, as used herein, may refer to sequence identity, or may refer to structural identity, or functional identity. By using the term “homology” and other like forms, it is to be understood that any molecule, whether nucleic acid or peptide, that functions similarly, and/or contains sequence identity, and/or is conserved structurally so that it approximates the reference sequence, is to be considered as part of this invention.

The term “homology”, as used herein, indicates a percentage of amino acid residues in the candidate sequence that are identical with the residues of a corresponding native polypeptide, after aligning the sequences and introducing gaps, if necessary, to achieve the maximum percent homology, and not considering any conservative substitutions as part of the sequence identity. Neither N- or C-terminal extensions nor insertions shall be construed as reducing identity or homology. Methods and computer programs for the alignment are well known in the art.

In one embodiment, the terms “homology”, “homologue” or “homologous”, in any instance, indicate that the sequence referred to, whether an amino acid sequence, or a nucleic acid sequence, exhibits at least 70% correspondence with the indicated sequence. In another embodiment, the amino acid sequence or nucleic acid sequence exhibits at least 72% correspondence with the indicated sequence. In another embodiment, the amino acid sequence or nucleic acid sequence exhibits at least 75% correspondence with the indicated sequence. In another embodiment, the amino acid sequence or nucleic acid sequence exhibits at least 77% correspondence with the indicated sequence. In another embodiment, the amino acid sequence or nucleic acid sequence exhibits at least 80% correspondence with the indicated sequence. In another embodiment, the amino acid sequence or nucleic acid sequence exhibits at least 82% correspondence with the indicated sequence. In another embodiment, the amino acid sequence or nucleic acid sequence exhibits at least 85% correspondence with the indicated sequence. In another embodiment, the amino acid sequence or nucleic acid sequence exhibits at least 87% correspondence with the indicated sequence. In another embodiment, the amino acid sequence or nucleic acid sequence exhibits at least 90% correspondence with the indicated sequence. In another embodiment, the amino acid sequence or nucleic acid sequence exhibits at least 92% correspondence with the indicated sequence. In another embodiment, the amino acid sequence or nucleic acid sequence exhibits at least 95% or more correspondence with the indicated sequence. In another embodiment, the amino acid sequence or nucleic acid sequence exhibits 95%-100% correspondence to the indicated sequence. In another embodiment, the amino acid sequence or nucleic acid sequence exhibits 100% correspondence to the indicated sequence. Similarly, as used herein, the reference to a correspondence to a particular sequence includes both direct correspondence, as well as homology to that sequence as herein defined.

It is to be understood that the polypeptides of this invention, and uses thereof, may comprise polypeptides which exhibit 100% identity with the described amino acid sequence, or in some embodiments, exhibiting less sequence identity, yet nonetheless, optimization of such polypeptide with a particular allele-restricted response is preserved, such that even when changes to the polypeptide sequence are introduced, the population is nonetheless responsive to the altered polypeptide.

In one embodiment, a polypeptide of this invention comprises at least 90% specificity for an HLA-DRB1 allele carried by a subject. In one embodiment, a polypeptide of this invention comprises at least 92% specificity for an HLA-DRB1 allele carried by a subject. In one embodiment, a polypeptide of this invention comprises at least 95% specificity for a HLA-DRB1 allele carried by a subject. In one embodiment, a polypeptide of this invention comprises at least 97% specificity for an HLA-DRB1 allele carried by a subject. In one embodiment, a polypeptide of this invention comprises 100% specificity for an HLA-DRB1 allele carried by a subject.

In one embodiment, HLA-DRB1 allele specificity of a polypeptide refers to ability of a particular polypeptide to stimulate an enhanced T cell response in assays as described herein.

In another embodiment, the “agent” encodes a piggyback molecule, enabling its entry into the brain, for example a brain specific interacting protein such as an antibody or a fragment thereof interacting specifically with brain-specific proteins.

In some embodiments, the compositions of the invention comprise an agent that increases brain levels of interferon-γ.

In some embodiments, the phrase “increases brain levels of interferon-γ” refers to a brain-exclusive phenomenon, such that brain levels are exclusively enhanced, or in some embodiments, preferentially enhanced, or in some embodiments, brain levels are enhanced, while peripheral levels are enhanced as well. In some embodiments, the phrase “increases brain levels of interferon-γ” refers to increased production of the interferon in the brain, or in some embodiments, increased production predominantly in the brain, or in some embodiments, increased production peripherally, which enables increased presence in the brain.

In one embodiment, the agent is interleukin-2 (IL-2). In another embodiment, the agent is IL-12. In another embodiment, the agent is IL-18. In another embodiment, the agent is interferon α or interferon β or tumor necrosis factor α. In another embodiment, the agent is lymphocyte growth hormone.

In another embodiment, the agent is derived from Brassica vegetables. In another embodiment, the agent is 3,3′-di-indolylmethane (DIM). In another embodiment, the agent is indole-3-carbimol.

In another embodiment, the agent is any compound, composition or agent which initiates a cell-mediated immune response in the brain.

In another embodiment, the agent is any compound, composition or agent which initiates a cell-mediated immune response in the central nervous system (CNS), which comprises the brain and the spinal cord. In another embodiment, the agent is any compound, composition or agent which initiates a cell-mediated immune response in the peripheral nervous system (PNS). In another embodiment, the agent is any compound, composition or agent which initiates a cell-mediated immune response in both the CNS and the PNS.

In some instances, it is desirable to determine the presence of interferon-γ in a subject, which can indicate, for example, whether the subject has an infection of the central nervous system. Examples of infections of the central nervous system include, for example, meningitis (bacterial or viral), encephalitis, polymyeloradiculitis, ventriculitis, myelitis, inflammatory polyneuropathy, meninigoencephalitis, acute cerebellar ataxia, aseptic meningitis, transverse myelitis, autonomic neuropathy, primary CNS lymphoma in AIDS, Bartonella henselae, Borrelia burgdorferi, Cryptococcal neoformas, Leptospira interrogans, Mycobacterium pneumoniae, Mycobacterium tuberculosis, Toxoplasma gondii, and Tophryma whippelii.

Interferon-γ can be measured, for example, from a sample of the cerebral spinal fluid, or serum more generally (e.g., serum from a blood sample of a patient). The interferon-γ can then be detected using standard assays such as an ELISA assay.

In one embodiment, the composition of the invention comprises an agent, which increases the number of T regulatory (Treg) cells, which in some embodiments, is specific for brain T regulatory cells.

T regulatory cells (Tregs) are a regulatory component of the immune system and act to modulate immune responses.

In some embodiments, the term “Tregs”, refers to a T cell population that inhibits or prevents the activation, or in another embodiment, the effector function, of another T lymphocyte. In one embodiment, the Tregs are a homogenous population, or in another embodiment, a heterogeneous population.

The Tregs of this invention express CD25 and CD4 on their cell surface. In one embodiment, the Tregs may be classified as CD25^{high} expressors, or in another embodiment, the Tregs may be classified as CD4^{low} expressors, or in another embodiment, a combination thereof. In another embodiment, the Tregs may express CTLA-4, or in another embodiment, GITR. In one embodiment, the Tregs may be classified as CTLA-4^{high} expressors, or in another embodiment, the Tregs may be classified as GITR^{high}, or in another embodiment, a combination thereof. In another embodiment, the Tregs of this invention are CD69−. In another embodiment, the Tregs of this invention are CD62L^{hi}, CD45RB^{lo}, CD45RO^{hi}, CD45RA−, αEβ7 integrin, Foxp3, expressors, or any combination thereof. It is to be understood that the agent, which increases the number of brain Tregs of this invention encompasses Treg cell populations expressing any number or combination of cell surface markers, as described herein, and as is well known in the art, and are to be considered as part of this invention.

In some embodiments, the phrase “T regulatory cells (Tregs)” refers to any cell population with such activity, for example, as described in U.S. Pat. No. 7,115,259, US Patent Application Publication No. US2005000244142, and US Patent Application Publication No. US20070172947A1.

In one embodiment, Tregs have unique cell surface expression profiles. In one embodiment, Tregs express CD4 or CD8, CD25 and Foxp3.

In one embodiment, the composition of the invention comprises an adjuvant.

An adjuvant comprises a substance that is added to a vaccine to improve the immune response. In some instances, the adjuvant can result in a lower necessary dose of vaccine required to produce sufficient quantity of antibodies. Such adjuvants can work by speeding the division of lymphocytes and by keeping the antigen in the area where the immune response is taking place. Some examples of adjuvants include alum, aluminum phosphate, aluminum hydroxide gel, QS21 and Freund's adjuvant (complete or incomplete).

In one embodiment, compositions of this invention are pharmaceutically acceptable. In one embodiment, the term “pharmaceutically acceptable” refers to any formulation which is safe, and provides the appropriate delivery for the desired route of administration of an effective amount of at least one compound for use in the present invention. This term refers to the use of buffered formulations as well, wherein the pH is maintained at a particular desired value, ranging from pH 4.0 to pH 9.0, in accordance with the stability of the compounds and route of administration.

In one embodiment, the composition can include a pharmaceutically acceptable carrier.

In one embodiment, the composition can include a carrier, diluent, lubricant, flow-aid or mixture thereof.

Solid carriers/diluents include, but are not limited to, a gum, a starch (e.g., corn starch, pregeletanized starch), a sugar (e.g., lactose, mannitol, sucrose, dextrose), a cellulosic material (e.g., microcrystalline cellulose), an acrylate (e.g., polymethylacrylate), calcium carbonate, magnesium oxide, talc, or mixtures thereof.

In one embodiment, a peptide of or used in the methods of this invention may be administered alone or within a composition. In another embodiment, compositions of this invention admixture with conventional excipients, i.e. pharmaceutically acceptable organic or inorganic carrier substances suitable for parenteral, enteral (e.g., oral) or topical application which do not deleteriously react with the active compounds may be used. In one embodiment, suitable pharmaceutically acceptable carriers include but are not limited to water, salt solutions, alcohols, gum arabic, vegetable oils, benzyl alcohols, polyethylene glycols, gelatine, carbohydrates such as lactose, amylose or starch, magnesium stearate, talc, silicic acid, viscous paraffin, white paraffin, glycerol, alginates, hyaluronic acid, collagen, perfume oil, fatty acid monoglycerides and diglycerides, pentaerythritol fatty acid esters, hydroxy methylcellulose, polyvinyl pyrrolidone, etc. In another embodiment, the pharmaceutical preparations can be sterilized and if desired mixed with auxiliary agents, e.g., lubricants, preservatives, stabilizers, wetting agents, emulsifiers, salts for influencing osmotic pressure, buffers, coloring, flavoring and/or aromatic substances and the like which do not deleteriously react with the active compounds. In another embodiment, they can also be combined where desired with other active agents, e.g., vitamins.

In one embodiment, pharmaceutical compositions of this invention comprise a peptide described herein or a pharmaceutically acceptable salt thereof, an adjuvant, and in some instance a pharmaceutically acceptable carrier or vehicle. The compositions delineated herein can also include additional therapeutic agents if present, in amounts effective for achieving a modulation of disease or disease symptoms, including disorders relating to accumulation of extracellular Aβ or symptoms thereof.

Pharmaceutically acceptable carriers, adjuvants and vehicles that may be used in the pharmaceutical compositions of this invention include, but are not limited to, ion exchangers, alum, alumina, aluminum stearate, lecithin, self-emulsifying drug delivery systems (SEDDS) such as d-a-tocopherol polyethyleneglycol 1000 succinate, surfactants used in pharmaceutical dosage forms such as Tweens or other similar polymeric delivery matrices, serum proteins, such as human serum albumin, buffer substances such as phosphates, glycine, sorbic acid, potassium sorbate, partial glyceride mixtures of saturated vegetable fatty acids, water, salts or electrolytes, such as protamine sulfate, disodium hydrogen phosphate, potassium hydrogen phosphate, sodium chloride, zinc salts, colloidal silica, magnesium trisilicate, polyvinyl pyrrolidone, cellulose-based substances, polyethylene glycol, sodium carboxymethylcellulose, polyacrylates, waxes, polyethylene-polyoxypropyle-ne-block polymers, polyethylene glycol and wool fat. Cyclodextrins such as .alpha.-, .beta.-, and .gamma.-cyclodextrin, may also be advantageously used to enhance delivery of compounds of the formulae described herein.

In one embodiment, the composition is in the form of a pellet, a tablet, a capsule, a solution, a suspension, a dispersion, an emulsion, an elixir, a gel, an ointment, a cream, or a suppository.

In one embodiment, the composition is in a form suitable for intracranial administration. In some embodiments, direct methods to introduce therapeutic agents into the brain substance include the use of devices and needles, such as in the case of intrathecal and intracerebroventricular delivery. In other embodiments, direct methods to introduce therapeutic agents into the brain substance include the use of magnets coupled to the composition of the invention for site-directed delivery. In other embodiments, direct methods to introduce therapeutic agents into the brain substance include the use of heat-activated compounds coupled to the composition of the invention for site-directed delivery. In one embodiment, delivery of the agent to the CNS is a function of its ability to access a relevant target site within the CNS.

In one embodiment, the composition is in a form suitable for intranasal administration. In some embodiments, intranasal delivery insures CNS delivery, upon crossing the olfactory nerves, the trigeminal nerves, or both. Intranasal delivery does not require any modification of the therapeutic agents and does not require that drugs be coupled with any carrier like in the case of drug delivery across the BBB. The olfactory neural pathway provides two pathways across the BBB. The intraneuronal pathway involves axonal transport and requires hours to days for drugs to reach different brain regions, while an extraneuronal pathway into the brain relies on bulk flow transport through perineural channels, which deliver drugs directly to the brain parenchymal tissue and/or CSF, and allows therapeutic agents to reach the CNS within minutes. In some embodiments, intranasal delivery is via the intraneuronal pathway. In other embodiments, intranasal delivery is via the extraneuronal pathway. In another embodiment, intransal delivery is via a combination of the intraneuronal and extraneuronal pathways.

For intranasal administration or application by inhalation, solutions or suspensions of the compounds mixed and aerosolized or nebulized in the presence of the appropriate carrier suitable. Such an aerosol may comprise any agent described herein.

In one embodiment, the composition is in a form suitable for oral, intravenous, intraarterial, intramuscular, subcutaneous, parenteral, transmucosal, transdermal, or topical administration.

In one embodiment, the route of administration may be parenteral, or a combination thereof. In another embodiment, the route may be intra-ocular, conjunctival, topical, transdermal, intradermal, subcutaneous, intraperitoneal, intravenous, intra-arterial, vaginal, rectal, intratumoral, parcanceral, transmucosal, intramuscular, intravascular, intraventricular, intracranial, inhalation (aerosol), nasal aspiration (spray), intranasal (drops), sublingual, oral, aerosol or suppository or a combination thereof.

For parenteral administration, particularly suitable are injectable, sterile solutions, preferably oily or aqueous solutions, as well as suspensions, emulsions, or implants, including suppositories and enemas. Ampoules are convenient unit dosages. Such a suppository may comprise any agent described herein.

In one embodiment, the pharmaceutical compositions are administered as a suppository, for example a rectal suppository or a urethral suppository. Further, in another embodiment, the pharmaceutical compositions are administered by subcutaneous implantation of a pellet. In a further embodiment, the pellet provides for controlled release of an agent over a period of time. In yet another embodiment, the pharmaceutical compositions are administered in the form of a capsule.

In one embodiment, the dosage regimen will be determined by skilled clinicians, based on factors such as exact nature of the condition being treated, the severity of the condition, the age and general physical condition of the patient, body weight, and response of the individual patient, etc.

In one embodiment, the pharmaceutical compositions provided herein are controlled release compositions, i.e. compositions in which the compound is released over a period of time after administration. Controlled- or sustained-release compositions include formulation in lipophilic depots (e.g., fatty acids, waxes, oils).

In one embodiment, the composition is an immediate-release composition, i.e. a composition in which the entire compound is released immediately after administration.

In one embodiment, the controlled or sustained release compositions of the invention are administered as a single dose. In another embodiment, compositions of the invention are administered as multiple doses, over a varying time period of minutes, hours, days, weeks, months or more. In another embodiment, compositions of the invention are administered during periods of acute disease. In another embodiment, compositions of the invention are administered during periods of chronic disease. In another embodiment, compositions of the invention are administered during periods of remission. In another embodiment, compositions of the invention are administered prior to development of gross symptoms.

In yet another embodiment, the pharmaceutical composition can be delivered in a controlled release system. For example, the agent may be administered using intravenous infusion, an implantable osmotic pump, a transdermal patch, liposomes, or other modes of administration. In one embodiment, a pump may be used. In another embodiment, polymeric materials can be used. In yet another embodiment, a controlled release system can be placed in proximity to the therapeutic target, i.e., the brain, thus requiring only a fraction of the systemic dose. In another embodiment, the controlled release system may be any controlled release system known in the art.

In one embodiment, the composition is a liquid dosage form. For liquid formulations, pharmaceutically acceptable carriers may be aqueous or non-aqueous solutions, suspensions, emulsions or oils. Examples of non-aqueous solvents are propylene glycol, polyethylene glycol, and injectable organic esters such as ethyl oleate. Aqueous carriers include water, alcoholic/aqueous solutions, emulsions or suspensions, including saline and buffered media. Examples of oils are those of petroleum, animal, vegetable, or synthetic origin, for example, peanut oil, soybean oil, mineral oil, olive oil, sunflower oil, and fish-liver oil.

In one embodiment, the composition is a solid dosage form.

In one embodiment, the invention features a method for eliciting an optimized immune response in a subject. In one embodiment this is an optimized T cell immune response in the central nervous system. The method includes administering to the patient any of the peptides or compositions described herein. In some embodiments, the immune response includes a reduction of Aβ accumulation in the central nervous system, for example the brain. The reduction of Aβ accumulation can occur, for example, by the clearance of Aβ via microglia or macrophages. In one embodiment, the immune response includes a reduction of Aβ accumulation is a subject. In some embodiments, the immune response is a T-cell response.

In one embodiment, a method of optimizing a T cell immune response comprises determining expression of a particular HLA-DRB1 allele in a sample derived from a subject and administering an optimized Aβ peptide to the subject, wherein the peptide carries an epitope which produces a positive T cell proliferation response in the subject carrying a particular HLA-DRB1 allele. The T-cell response can be determined in a variety of ways, including by exposing the peptide to PBMC. In some embodiments, the T cell response is in the central nervous system, for example in the brain.

In one embodiment, a polypeptide used in the methods of this invention has an amino acid sequence, which corresponds to or is homologous to any one of SEQ ID NOs: 1-15, 17-34, as shown in Table 1.

In one embodiment, this invention provides a method of optimized treatment of a subject afflicted with a disease or disorder associated with amyloid beta accumulation, wherein the method comprises determining expression of a particular HLA-DRB1 allele in a sample derived from a subject and administering an optimized peptide to the subject, whereby administration of the optimized peptide to the subject expressing a particular HLA-DRB1 allele results in an optimal treatment for the subject.

In one embodiment, optimal treatment refers to a treatment in which an immune response is elicited due to method of use in which a particular polypeptide is administered to a subject such that the polypeptide comprises an HLA-DRB1 specific epitope of the HLA-DRB1 allele carried by the subject.

In one embodiment, this invention provides a method of optimized immunization against a disease characterized by amyloid beta accumulation in a subject, wherein the method comprises determining expression of a particular HLA-DRB1 allele in a sample derived from a subject and administering an optimized peptide to the subject, whereby, administration of the optimized peptide to said subject expressing a particular HLA-DRB1 allele reduces a risk of disease characterized by amyloid beta accumulation or reduces severity of disease characterized by amyloid beta accumulation for said subject.

In one embodiment, the methods of this invention provide that if a subject expresses an HLA-DRB1 1501 allele, then the peptide administered to the subject has an amino acid sequence as set forth in any one of SEQ ID NOs: 1, 2, 7, 14, 20, 21, 27-34, or any combination thereof.

In one embodiment, the methods of this invention provide that if a subject expresses an HLA-DRB1 0301 allele, then the peptide administered to the subject has an amino acid sequence as set forth in SEQ ID NOs: 3, 4, 5, 6 or 7, or any combination thereof.

In one embodiment, the methods of this invention provide that if a subject expresses an HLA-DRB1 0101 allele, then the peptide administered to the subject has an amino acid sequence as set forth in any one of SEQ ID NOs: 3, 4, 5, 7, 8, 22-28 or any combination thereof.

In one embodiment, the methods of this invention provide that if a subject expresses an HLA-DRB1 1301 allele, then the peptide administered to the subject has an amino acid sequence as set forth in SEQ ID NOs: 7, 9, 10, 24, 25, or 26, or any combination thereof.

In one embodiment, the methods of this invention provide that if a subject expresses an HLA-DRB1 1502 allele, then the peptide administered to the subject has an amino acid sequence as set forth in SEQ ID NOs: 3, 4, 5 or 7, or any combination thereof.

In one embodiment, the methods of this invention provide that if a subject expresses an HLA-DRB1 0404 allele, then the peptide administered to the subject has an amino acid sequence as set forth in SEQ ID NOs: 7, 9, 10, 11, 19, 24, 25, or 26, or any combination thereof.

In one embodiment, the methods of this invention provide that if a subject expresses an HLA-DRB1 1001 allele, then the peptide administered to the subject has an amino acid sequence as set forth in SEQ ID NOs: 7, 12, 13, 22, 23, or 24, or any combination thereof.

In one embodiment, the methods of this invention provide that if a subject expresses an HLA-DRB1 0401 allele, then the peptide administered to the subject has an amino acid sequence as set forth in SEQ ID NOs: 7, 9, 11, 13, 15, 18, 22, 23, or 24, or any combination thereof.

In one embodiment, the methods of this invention provide optimized treatment of a Caucasian subject afflicted with a disease or disorder associated with amyloid beta accumulation, the method comprising, administering at least one polypeptide to the Caucasian subject, wherein the polypeptide has an amino acid sequence as set forth in any one of SEQ ID NOs: 1-7, 14, 20-34 or a combination thereof. In one embodiment, the Caucasian subject is at risk for a neurodegenerative disease or disorder.

In one embodiment of this invention, “Caucasian” refers to a white individual with Western European ancestral origins.

In one embodiment, the methods of this invention provide optimized treatment of an Asian or an Arab subject afflicted with a disease or disorder associated with amyloid beta accumulation, the method comprising, administering at least one polypeptide to the Asian or Arab subject, wherein the polypeptide has an amino acid sequence as set forth in any one of SEQ ID NOs: 1-7, 9, 11-15, 18, 20-24, 27-34, or a combination thereof. In one embodiment, the Asian or Arab subject is at risk for a neurodegenerative disease or disorder.

In one embodiment of this invention, “Asian refers to an individual with Chinese, Japanese, and/or Korean ancestral origins. In one embodiment of this invention, “Arab” refers to an individual with Lenanese and/or Jordanian ancestral origins.

In one embodiment, the methods of this invention provide optimized treatment of an African subject afflicted with a disease or disorder associated with amyloid beta accumulation, the method comprising, administering at least one polypeptide to the African subject, wherein the polypeptide has an amino acid sequence as set forth in SEQ ID NOs: 3-7, 9, 10, 12, 13, 22, 23, or 24, or a combination thereof. In one embodiment, the African subject is at risk for a neurodegenerative disease or disorder.

In one embodiment of this invention, “African” refers to a black individual with African ancestral origins.

In one embodiment, the methods of this invention provide optimized treatment of an African American subject afflicted with a disease or disorder associated with amyloid beta accumulation, the method comprising, administering at least one polypeptide to the African American subject, wherein the polypeptide has an amino acid sequence as set forth in SEQ ID NOs: 3-7, 12, 13, 22, 23, or 24, or a combination thereof. In one embodiment, the African American subject is at risk for a neurodegenerative disease or disorder.

In one embodiment of this invention, “African American” refers to an individual with ancestral origins from both Africa and The United States.

In one embodiment, this invention provides a method of optimized treatment of a subject afflicted with a disease or disorder associated with amyloid beta accumulation, wherein the method comprises determining expression of a particular HLA-DRB1 allele in a sample derived from a subject and if the subject carries a particular allele then the subject will be responsive to an amyloid beta peptide-dependent regimen.

In one embodiment, this invention provides a method of optimized immunization against a disease characterized by amyloid beta accumulation in a subject, wherein the method comprises determining expression of a particular HLA-DRB1 allele in a sample derived from a subject and if the subject carries a particular allele then the subject will be responsive to an amyloid beta peptide-dependent immunization regimen.

In one embodiment, the methods of this invention provide that if a subject expresses an HLA-DRB1 1501 allele, an HLA-DRB1 0301, an HLA-DRB1 0101 allele, an HLA-DRB1 1301 allele, an HLA-DRB1 1502 allele, an HLA-DRB1 0404 allele, an HLA-DRB1 1001 allele, an HLA-DRB1 0401 allele, an HLA-DRB1 1104 allele, an HLA-DRB1 0402 allele, an HLA-DRB1 04011 allele, an HLA-DRB1 1302 allele, an HLA-DBR1 0403 allele, a HLA-DRB1 1102 allele, an HLA-DRB1 0103 allele, an HLA-DRB1 0407 allele, an HLA-DRB1 0302 allele, or an HLA-DRB1 1404 allele, then the subject will be responsive to an amyloid beta peptide-dependent treatment and/or immunization regimen.

In one embodiment, the invention provides a method of optimized treatment of a subject afflicted with a disease or disorder associated with Aβ accumulation, by administering to the subject any of the peptides or compositions described herein. Any suitable route of administration can be used.

In one embodiment, the invention provides a method of optimized immunization against a disease or disorder characterized by Aβ accumulation in a subject, by administering to the subject any of the peptides or compositions described herein. Any suitable route of administration can be used.

In one embodiment, the invention provides a method for optimizing treatment of a subject afflicted with a disease or disorder characterized by Aβ accumulation, the method including administering to the subject any of the peptides or compositions described herein. In one embodiment, a method of optimizing treatment of a subject includes identifying an HLA-DRB1 allele of a subject, selecting an optimized polypeptide of this invention and administering to the identified subject a composition including an optimized peptide of this invention. In some embodiments, an optimized peptide comprises a T cell epitope. In some embodiments, an optimized peptide comprises a B cell epitope. In some embodiments, an optimized peptide comprises a T cell and a B cell epitope. In some embodiments, the peptide is administered as part of a composition. In some embodiments, the composition includes an agent which increases interferon-γ. In some embodiments, the composition includes an agent which increases Treg cells. In some embodiments, the methods includes identifying that the subject as free of infection in the central nervous system. In some instances, a subject is identified as free of infection in the central nervous system by being substantially free of interferon-γ in the central nervous system, for example by taking a sample of cerebral spinal fluid and measuring for the presence of interferon-γ using an ELISA assay. In another embodiment, the method also includes isolating PBMC from a subject; exposing the PBMC to an optimized of Aβ peptide; and measuring the T cell proliferation response. In some embodiments, the method also includes measuring the B cell response.

In another aspect, the invention includes a method for determining an adverse reaction to a treatment with an Aβ1-42 antigen in a subject, the method includes identifying an HLA-DRB1 allele of a subject and matching the identified HLA-DRB1 allele to any of the alleles shown to be non-responsive to Aβ treatment, as described herein.

In some cases it is beneficial to monitor the immune response of a subject over time. For example, the relative increase Aβ reactive T-cells.

It is also beneficial to monitor the subject's immune response, such as a T cell response and/or B cell response, before and after the administration of an Aβ vaccine or Aβ optimized treatment.

The subject's immune response can be measured, for example, using the techniques described herein, such as measuring cytokine production or by measuring the presence of antibody in serum.

For example, a subject can provide a blood sample at an initial time where the immune response is determined. This initial determination can then be compared with the same subject's immune response at a second time, e.g., one week, one month, two months, three months, or six months later.

The peptides of this invention can be made by chemical synthesis methods, which are well known, to the ordinarily skilled artisan. See, for example, Fields et al., Chapter 3 in Synthetic Peptides: A User's Guide, ed. Grant, W. H. Freeman & Co., New York, N.Y., 1992, p. 77. Hence, peptides can be synthesized using the automated Merrifield techniques of solid phase synthesis with the .alpha.-NH2 protected by either t-Boc or F-moc chemistry using side chain protected amino acids on, for example, an Applied Biosystems Peptide Synthesizer Model 430A or 431.

For example, methods of making peptides are well known in the art. One manner of making of the peptides described herein is using solid phase peptide synthesis (SPPS). The C-terminal amino acid is attached to a cross-linked polystyrene resin via an acid labile bond with a linker molecule. This resin is insoluble in the solvents used for synthesis, making it relatively simple and fast to wash away excess reagents and by-products. The N-terminus is protected with the Fmoc group, which is stable in acid, but removable by base. Any side chain functional groups are protected with base stable, acid labile groups.

Alternatively, the longer synthetic peptides can be synthesized by well known recombinant DNA techniques. Such techniques are provided in well-known standard manuals with detailed protocols. To construct a gene encoding a peptide of this invention, the amino acid sequence is reverse translated to obtain a nucleic acid sequence encoding the amino acid sequence, preferably with codons that are optimum for the organism in which the gene is to be expressed. Next, a synthetic gene is made, typically by synthesizing oligonucleotides which encode the peptide and any regulatory elements, if necessary. The synthetic gene is inserted in a suitable cloning vector and transfected into a host cell. The peptide is then expressed under suitable conditions Appropriate for the selected expression system and host. The peptide is purified and characterized by standard methods.

In one embodiment, the invention provides methods for treating a subject having, a neurodegenerative disease or disorder. In one embodiment the neurodegenerative disease or disorder comprises an injury, disease, disorder or condition of the central nervous system (CNS). In one embodiment, the neurodegenerative disease or disorder comprises Alzheimer's disease, Parkinson's disease, Huntington's disease, multiple sclerosis, diabetic neuropathy or amyotrophic lateral sclerosis (ALS). In one embodiment, the neurodegenerative disease or disorder comprises spinal cord injury, closed head injury, blunt trauma, penetrating trauma, hemorrhagic stroke, ischemic stroke, cerebral ischemia, optic nerve injury, or injury caused by tumor excision.

In one embodiment, the subject is at risk for a neurodegenerative disease or disorder.

In one embodiment, the invention provides methods for treating a subject having a renal disease or disorder. In one embodiment, the renal disease or disorder comprises an injury, disease, disorder or condition of the kidneys. In one embodiment, the renal disease or disorder comprises renal amyloidosis, hereditary renal amyloidosis, or dialysis-associated arthropathy.

In one embodiment, the subject is at risk for a renal disease or disorder.

In one embodiment, the invention provides methods for treating a subject having a muscle disease or disorder. In one embodiment, the muscle disease or disorder comprises an injury, disease, disorder or condition of the muscles. In one embodiment, the muscle disease or disorder comprises inclusion body myositis.

In one embodiment, the subject is at risk for a muscle disease or disorder.

In one embodiment, the invention provides methods for treating a subject having a skin disease or disorder. In one embodiment, the skin disease or disorder comprises an injury, disease, disorder or condition of skin. In one embodiment, the skin disease or disorder comprises cutaneous amyloidosis.

In one embodiment, the subject is at risk for a skin disease or disorder.

In one embodiment, the invention provides methods for treating a subject having a cardiac disease or disorder. In one embodiment, the cardiac disease or disorder comprises an injury, disease, disorder or condition of the heart. In one embodiment, the cardiac disease or disorder comprises cardiac amyloidosis.

In one embodiment, the subject is at risk for a cardiac disease or disorder.

In one embodiment, the invention provides methods for treating a subject having an amyloidosis disease or disorder. In one embodiment, the amyloidosis disease or disorder comprises AL-amyloidosis, AA-amyloidosis, haemodialysis-associated amyloidosis, Finnish type amyloidosis, Familial Mediterranean Fever, or systemic amyloidosis.

In one embodiment, the subject is at risk for an amyloidosis disease or disorder.

In one embodiment, the invention provides a method of optimized treatment of a subject afflicted with an amyloid fibril disorder. In one embodiment, the invention provides a method of optimized immunization against a disease characterized by an amyloid fibril disorder in a subject. The methods include administering to the subject any of the peptides or compositions described herein. The amyloid fibril disorder can include a form of dementia (e.g., Alzheimer's disease). Other amyloid fibril disorders amenable to treatment by the methods described herein include Down's Syndrome, Dutch Type Hereditary Cerebral Hemorrhage Amyloidosis, Reactive Amyloidosis, Familial Mediterranean Fever, Familial Amyloid Nephropathy with Urticaria and Deafness, Muckle-Wells Syndrome, Idiopathic Myeloma; Macroglobulinemia-Associated Myeloma, Familial Amyloid Polyneuropathy, Familial Amyloid Cardiomyopathy, Isolated Cardiac Amyloid, Systemic Senile Amyloidosis, Adult Onset Diabetes, Insulinoma, Isolated Atrial Amyloid, Medullary Carcinoma of the Thyroid, Familial Amyloidosis, Hereditary Cerebral Hemorrhage with Amyloidosis, Familial Amyloidotic Polyneuropathy, Scrapie, Creutzfeldt-Jacob Disease, Gerstmann Straussler-Scheinker Syndrome, Bovine Spongiform Encephalitis, a Prion-mediated disease, or Huntington's Disease.

In some embodiments of this invention, the route of administration will be determined by skilled clinicians, based on factors such as the exact nature of the condition being treated, the severity of the condition, the age and general physical condition of the subject, body weight, and response of the individual subject, and combinations thereof. For example, in one embodiment, a route of administration for a subject afflicted with a neurodegenerative disease or disorder may be intracranial. In one embodiment, a route of administration for a subject afflicted with a neurodegenerative disease or disorder may be intranasal. In one embodiment, a route of administration for a subject afflicted with a renal disease or disorder may be parenteral. In one embodiment, a route of administration for a subject afflicted with a renal disease or disorder may be oral. In one embodiment, a route of administration for a subject afflicted with a muscle disease or disorder may be parenteral. In one embodiment, a route of administration for a subject afflicted with a muscle disease or disorder may be oral. In one embodiment, a route of administration for a subject afflicted with a skin disease or disorder may be topical. In one embodiment, a route of administration for a subject afflicted with a muscle disease or disorder may be oral. In one embodiment, a route of administration for a subject afflicted with a cardiac disease or disorder may be parenteral. In one embodiment, a route of administration for a subject afflicted with a cardiac disease or disorder may be oral.

In some embodiment, the dosage regimen will be determined by skilled clinicians, based on factors such as exact nature of the condition being treated, the severity of the condition, the age and general physical condition of the subject, body weight, and response of the individual subject, and combinations thereof. For example, in some embodiments dosage may be increased or decreased based on the response of the individual.

In one embodiment, the methods of this invention provide pharmaceutical compositions formulated for an organ-specific delivery. In one embodiment, the organ-specific delivery comprises brain-specific delivery, kidney-specific delivery, heart-specific delivery, cutaneous-specific delivery, or muscle-specific delivery, or any combination thereof.

In one embodiment, the peptides and compositions this invention can be administered in a variety of manners. For example, the compound can be administered orally, parenterally, or mucosally (e.g., nasally).

In one embodiment, the invention provides a method of optimized treatment of a subject by administering to the subject any of the peptides or compositions described herein. Any suitable route of administration can be used. In one embodiment, the methods of this invention provide for administration via an intracranial route. In one embodiment, the methods provide for administration via an intranasal route. In one embodiment, the methods provide for administration via an oral, intravenous, intraarterial, intramuscular, subcutaneous, parenteral, transmucosal, transdermal, or topical route.

In one embodiment, the invention includes a method for determining an adverse reaction to an immunization with an Aβ antigen in a patient.

In one embodiment, this invention provides a method for assessing responsiveness of a subject to an amyloid beta peptide-dependent treatment regimen, the method comprising, determining expression of a particular HLA-DRB1 allele in a sample derived from a subject, wherein if the subject expresses any two of the following alleles, an HLA-DRB1 1101 allele, an HLA-DRB1 0801 allele, an HLA-DRB1 0102 allele, an HLA-DRB1 00170 allele or an HLA-DRB1 0701 allele,then the subject is considered to be non-responsive to an amyloid beta peptide-dependent treatment regimen.

This invention encompasses administration of peptides as described herein or compositions comprising the same, for treating subjects afflicted with diseases and disorders related to accumulation of Aβ or immunizing subjects against a disease characterized by Aβ accumulation in a subject.

Delivery of peptides or compositions of this invention to the CNS may, in some embodiments of this invention, be by systemic administration, injection into CSF pathways, or direct injection into the brain, and in some embodiments, the compositions of this invention are formulated for any of these routes. In one embodiment, the compositions of the present invention are administered by systemic or direct administration into the CNS for targeted action in the CNS, and in some embodiments, the compositions of this invention are formulated for any of these routes. In one embodiment, the composition as set forth herein is formulated for brain-specific delivery, and in some embodiments, the compositions of this invention are formulated for any of these routes. In one embodiment, strategies for drug delivery to the brain include osmotic and chemical opening of the blood-brain barrier (BBB), as well as the use of transport or carrier systems, enzymes, and receptors that control the penetration of molecules in the blood-brain barrier endothelium, and in some embodiments, the compositions of this invention are formulated for any of these routes. In another embodiment, receptor-mediated transcytosis can transport peptides and proteins across the BBB, and in some embodiments, the compositions of this invention are formulated for any of these routes. In other embodiments, strategies for drug delivery to the brain involve bypassing the BBB, and in some embodiments, the compositions of this invention are formulated for any of these routes. In some embodiments, various pharmacological agents are used to open the BBB, and in some embodiments, the compositions of this invention are formulated for any of these routes.

In one embodiment, the route of administration may be directed to an organ or system that is affected by neurodegenerative conditions. For example, compounds may be administered topically. In another embodiment, the route of administration may be directed to a different organ or system than the one that is affected by neurodegenerative conditions. For example, compounds may be administered parenterally to treat neurodegenerative conditions. Thus, the present invention provides for the use of various dosage forms suitable for administration using any of the routes listed herein, and any routes which avail the CNS of such materials, as will be appreciated by one skilled in the art.

In some embodiments, the compositions/agents of the invention are specifically formulated such that they cross the blood-brain barrier (BBB). One example of such formulation comprises the use of specialized liposomes, which may be manufactured, for example as described U.S. Pat. Nos. 4,522,811; 5,374,548; and 5,399,331. In some embodiments, the liposomes comprise one or more moieties which are selectively transported into specific cells or organs (“targeting moieties” or “targeting groups” or “transporting vectors”), thus providing targeted drug delivery (see, e.g., V. V. Ranade J. Clin. Phamacol. 29, 685 (1989) fully incorporated by reference herein). In some embodiments the agents are linked to targeting groups that facilitate penetration of the blood brain barrier. In some embodiments, to facilitate transport of agents of the invention across the BBB, they may be coupled to a BBB transport vector (see, for example, Bickel et al., Adv. Drug Delivery Reviews 46, 247-79 (2001) fully incorporated by reference herein). In some embodiments, transport vectors include cationized albumin or the OX26 monoclonal antibody to the transferrin receptor; which undergo absorptive-mediated and receptor-mediated transcytosis through the BBB, respectively. Natural cell metabolites that may be used as targeting groups include, inter alia, putrescine, spermidine, spermine, or DHA. Other exemplary targeting moieties include folate or biotin (see, e.g., U.S. Pat. No. 5,416,016 fully incorporated by reference herein); mannosides (Umezawa et al., Biochem. Biophys. Res. Commun. 153, 1038 (1988) fully incorporated by reference herein); antibodies (P. G. Bloeman et al., FEBS Lett. 357, 140 (1995); M. Owais et al., Antimicrob. Agents Chemother. 39, 180 (1995)); surfactant protein A receptor (Briscoe et al., Am. J. Physiol. 1233, 134 (1995 fully incorporated by reference herein)); gp120 (Schreier et al., J. Biol. Chem. 269, 9090 (1994)); see also, K. Keinanen and M. L. Laukkanen, FEBS Lett. 346, 123 (1994); J. J. Killion and I. J. Fidler, Immunomethods 4, 273 (1994) all of which are fully incorporated by reference herein).

In some embodiments, BBB transport vectors that target receptor-mediated transport systems into the brain comprise factors such as insulin, insulin-like growth factors (“IGF-I,” and “IGF-II”), angiotensin II, atrial and brain natriuretic peptide (“ANP,” and “BNP”), interleukin I (“IL-1”) and transferrin. Monoclonal antibodies to the receptors that bind these factors may also be used as BBB transport vectors. BBB transport vectors targeting mechanisms for absorptive-mediated transcytosis include cationic moieties such as cationized LDL, albumin or horseradish peroxidase coupled with polylysine, cationized albumin or cationized immunoglobulins. Small basic oligopeptides such as the dynorphin analogue E-2078 and the ACTH analogue ebiratide may also cross the brain via absorptive-mediated transcytosis and are potential transport vectors. Other BBB transport vectors target systems for transporting nutrients into the brain. Examples of such BBB transport vectors include hexose moieties, e.g., glucose and monocarboxylic acids, e.g., lactic acid and neutral amino acids, e.g., phenylalanine and amines, e.g., choline and basic amino acids, e.g., arginine, nucleosides, e.g., adenosine and purine bases, e.g., adenine, and thyroid hormone, e.g., triiodothyridine. Antibodies to the extracellular domain of nutrient transporters may also be used as transport vectors. Other possible vectors include angiotensin II and ANP, which may be involved in regulating BBB permeability.

In some cases, the bond linking the therapeutic agent to the transport vector may be cleaved following transport into the brain in order to liberate the biologically active agent. Exemplary linkers include disulfide bonds, ester-based linkages, thioether linkages, amide bonds, acid-labile linkages, and Schiff base linkages. Avidin/biotin linkers, in which avidin is covalently coupled to the BBB drug transport vector, may also be used. Avidin itself may be a drug transport vector. Transcytosis, including receptor-mediated transport of compositions across the blood brain barrier, may also be suitable for the agents of the invention. Transferrin receptor-mediated delivery is disclosed in U.S. Pat. Nos. 5,672,683; 5,383,988; 5,527,527; 5,977,307; and 6,015,555, all of which are fully incorporated herein by reference. Transferrin-mediated transport is also known. P.M. Friden et al., Pharmacol. Exp. Ther. 278, 1491-98 (1996); H. J. Lee, J. Pharmacol. Exp. Ther. 292, 1048-52 (2000) all of which are fully incorporated herein by reference. EGF receptor-mediated delivery is disclosed in Y. Deguchi et al., Bioconjug. Chem. 10, 32-37 (1999), and transcytosis is described in A. Cerletti et al., J. Drug Target. 8, 435-46 (2000) all of which are fully incorporated herein by reference. Insulin fragments have also been used as carriers for delivery across the blood brain barrier. M. Fukuta et al., Pharm. Res. 11. 1681-88 (1994). Delivery of agents via a conjugate of neutral avidin and cationized human albumin has also been described. Y. S. Kang et al., Pharm. Res. 1, 1257-64 (1994) all of which are fully incorporated herein by reference. Other modifications in order to enhance penetration of the agents of the invention across the blood brain barrier may be accomplished using methods and derivatives known in the art. For example, U.S. Pat. No. 6,024,977 discloses covalent polar lipid conjugates for targeting to brain and central nervous system. U.S. Pat. No. 5,017,566 discloses cyclodextrin derivatives comprising inclusion complexes of lipoidal forms of dihydropyridine redox targeting moieties. U.S. Pat. No. 5,023,252 discloses the use of pharmaceutical compositions comprising a neurologically active drug and a compound for facilitating transport of the drug across the blood-brain barrier including a macrocyclic ester, diester, amide, diamide, amidine, diamidine, thioester, dithioester, thioamide, ketone or lactone. U.S. Pat. No. 5,024,998 discloses parenteral solutions of aqueous-insoluble drugs with cyclodextrin derivatives. U.S. Pat. No. 5,039,794 discloses the use of a metastatic tumor-derived egress factor for facilitating the transport of compounds across the blood-brain barrier. U.S. Pat. No. 5,112,863 discloses the use of N-acyl amino acid derivatives as antipsychotic drugs for delivery across the blood-brain barrier. U.S. Pat. No. 5,124,146 discloses a method for delivery of therapeutic agents across the blood-brain barrier at sites of increase permeability associated with brain lesions. U.S. Pat. No. 5,153,179 discloses acylated glycerol and derivatives for use in a medicament for improved penetration of cell membranes. U.S. Pat. No. 5,177,064 discloses the use of lipoidal phosphonate derivatives of nucleoside antiviral agents for delivery across the blood-brain barrier. U.S. Pat. No. 5,254,342 discloses receptor-mediated transcytosis of the blood-brain barrier using the transferrin receptor in combination with pharmaceutical compounds that enhance or accelerate this process. U.S. Pat. No. 5,258,402 discloses treatment of epilepsy with imidate derivatives of anticonvulsive sulfamate. U.S. Pat. No. 5,270,312 discloses substituted piperazines as central nervous system agents. U.S. Pat. No. 5,284,876 discloses fatty acid conjugates of dopamine drugs. U.S. Pat. No. 5,389,623 discloses the use of lipid dihydropyridine derivatives of anti-inflammatory steroids or steroid sex hormones for delivery across the blood-brain barrier. U.S. Pat. No. 5,405,834 discloses prodrug derivatives of thyrotropin releasing hormone. U.S. Pat. No. 5,413,996 discloses acyloxyalkyl phosphonate conjugates of neurologically-active drugs for anionic sequestration of such drugs in brain tissue. U.S. Pat. No. 5,434,137 discloses methods for the selective opening of abnormal brain tissue capillaries using bradykinin infused into the carotid artery. U.S. Pat. No. 5,442,043 discloses a peptide conjugate between a peptide having a biological activity and incapable of crossing the blood-brain barrier and a peptide which exhibits no biological activity and is capable of passing the blood-brain barrier by receptor-mediated endocytosis. U.S. Pat. No. 5,466,683 discloses water soluble analogues of an anticonvulsant for the treatment of epilepsy. U.S. Pat. No. 5,525,727 discloses compositions for differential uptake and retention in brain tissue comprising a conjugate of a narcotic analgesic and agonists and antagonists thereof with a lipid form of dihydropyridine that forms a redox salt upon uptake across the blood-brain barrier that prevents partitioning back to the systemic circulation all of which are fully incorporated herein by reference.

It is to be understood that reference to any publication, patent application or issued patent is to be considered as fully incorporated herein by reference in its entirety.

Nitric oxide is a vasodilator of the peripheral vasculature in normal tissue of the body. Increasing generation of nitric oxide by nitric oxide synthase causes vasodilation without loss of blood pressure. The blood-pressure-independent increase in blood flow through brain tissue increases cerebral bioavailability of blood-born compositions. This increase in nitric oxide may be stimulated by administering L-arginine. As nitric oxide is increased, cerebral blood flow is consequently increased, and drugs in the blood stream are carried along with the increased flow into brain tissue. Therefore, L-arginine may be used in the pharmaceutical compositions of the invention to enhance delivery of agents to brain tissue after introducing a pharmaceutical composition into the blood stream of the subject substantially contemporaneously with a blood flow enhancing amount of L-arginine, as described in WO 00/56328.

Still further examples of modifications that enhance penetration of the blood brain barrier are described in International (PCT) Application Publication Number WO 85/02342, which discloses a drug composition comprising a glycerolipid or derivative thereof. PCT Publication Number WO 089/11299 discloses a chemical conjugate of an antibody with an enzyme which is delivered specifically to a brain lesion site for activating a separately-administered neurologically-active prodrug. PCT Publication Number WO 91/04014 discloses methods for delivering therapeutic and diagnostic agents across the blood-brain barrier by encapsulating the drugs in liposomes targeted to brain tissue using transport-specific receptor ligands or antibodies. PCT Publication Number WO 91/04745 discloses transport across the blood-brain barrier using cell adhesion molecules and fragments thereof to increase the permeability of tight junctions in vascular endothelium. PCT Publication Number WO 91/14438 discloses the use of a modified, chimeric monoclonal antibody for facilitating transport of substances across the blood-brain barrier. PCT Publication Number WO 94/01131 discloses lipidized proteins, including antibodies. PCT Publication Number WO 94/03424 discloses the use of amino acid derivatives as drug conjugates for facilitating transport across the blood-brain barrier. PCT Publication Number WO 94/06450 discloses conjugates of neurologically-active drugs with a dihydropyridine-type redox targeting moiety and comprising an amino acid linkage and an aliphatic residue. PCT Publication Number WO 94/02178 discloses antibody-targeted liposomes for delivery across the blood-brain barrier. PCT Publication Number WO 95/07092 discloses the use of drug-growth factor conjugates for delivering drugs across the blood-brain barrier. PCT Publication Number WO 96/00537 discloses polymeric microspheres as injectable drug-delivery vehicles for delivering bioactive agents to sites within the central nervous system. PCT Publication Number WO 96/04001 discloses omega-3-fatty acid conjugates of neurologically-active drugs for brain tissue delivery. PCT WO 96/22303 discloses fatty acid and glycerolipid conjugates of neurologically-active drugs for brain tissue delivery. In one embodiment, the active compound can be delivered in a vesicle, for example, a liposome. In another embodiment, the active compound can be delivered as a nanoparticle. In one embodiment, delivery may be specifically targeted to the CNS. In another embodiment, the active compounds may be delivered by any method described herein. The compositions of this invention may comprise ingredients known to the skilled artisan to be useful in formulating compositions for administration to a subject. In some embodiments, the compositions will comprise pharmaceutically acceptable carriers or diluents. In some embodiments, the phrase “pharmaceutically acceptable carriers or diluents” may comprise a solid carrier or diluent for solid formulations, a liquid carrier or diluent for liquid formulations, or mixtures thereof.

In some embodiments, the compositions/agents of the invention comprise a “piggyback mechanism” to deliver specific desirable agents, or combinations thereof to the CNS, i.e. to ensure that they cross the blood-brain barrier (BBB).

In one embodiment, the compositions described herein can, for example, be administered by injection, intravenously, intraarterially, subdermally, intraperitoneally, intramuscularly, or subcutaneously; or orally, buccally, nasally, transmucosally, topically, in an ophthalmic preparation, or by inhalation. The methods herein contemplate administration of an effective amount of compound or compound composition to achieve the desired or stated effect. The amount of active ingredient that may be combined with the carrier materials to produce a single dosage form will vary depending upon the host treated and the particular mode of administration.

In one embodiment of this invention, “effective amount” refers to the amount of a composition required to be administered to a subject that induces a desired response. Some examples of effective amounts include the amount of a pharmaceutical composition required to alleviate a symptom such as pain or inflammation, or the amount of a composition required to induce an immune response in a subject, e.g., a vaccine. An effective amount can be determined using objective factors such as a measurable reduction in inflammation, or an effective amount can be measured subjectively, for example, based on a subject's description of a change in a symptom such as pain.

Specific dosage and treatment regimens for any particular patient will depend upon a variety of factors, including the activity of the specific composition employed, the age, body weight, general health status, sex, diet, time of administration, rate of excretion, drug combination, the severity and course of the disease, condition or symptoms, the patient's disposition to the disease, condition or symptoms, and the judgment of the treating physician.

In one embodiment, methods of the present invention involve treating a subject by, inter alia, controlling the expression, production, and activity of cytokines, chemokines and interleukins; anti-oxidant therapy; anti-endotoxin therapy or any combination thereof.

In one embodiment, the methods of the present invention may be used in animal models. Animal models representing a range of amyloidosis may provide an analysis tool for efficacy of polypeptides of this invention, polypeptides and compositions used in the methods of this invention, routes of administration of such, dosages of such, and treatment regimens. Results of animal models presented in U.S. patent application No. 61/008,580 and Monsonego et al., PNAS 103:, 5048-5053 (2006), are thereby incorporated by reference as if fully set forth herein.

The administration mode of the compounds and compositions of the present invention, timing of administration and dosage, i.e. the treatment regimen, will depend on the type and severity of the injury, disease or disorder, and the age and condition of the subject, and will be determined by skilled clinicians in the art. In one embodiment, the compounds and compositions may be administered concomitantly. In another embodiment, the compounds and compositions may be administered at time intervals of seconds, minutes, hours, days, weeks or more.

In one embodiment, treatment of a subject may involve different routes of administration, for example treatment of a subject afflicted with neurological disease or disorders may involve intracranial and intra nasal routes of administration. In one embodiment, treatment of a subject may involve multiple exposures to polypeptides and/or compositions comprising polypeptides of this invention. In one embodiment, exposures may be on a regular basis. In one embodiment, an exposure regimen will be determined by the skilled clinician based on response of a subject and may change over time.

It would be recognized by one skilled in the art, that an HLA-DRB1 allele may responds to multiple epitopes. The Examples described hereinbelow demonstrate that a range of peptides may specifically stimulate a positive T cell proliferation response in an HLA-DRB1 restricted cell line. Therefore, in one embodiment, treatment may involve compositions comprising differing peptides of this invention, each of which shows epitope specificity for a particular HLA-DRB1 allele. In some embodiment, methods of this invention use at least a single peptide from SEQ ID No. 1-15, 17-34 for optimized treatment of a subject afflicted with a disease or disorder characterized by Aβ accumulation. In some embodiment, methods of this invention use at least a single peptide from SEQ ID No. 1-15, 17-34 for optimized immunization against a disease or disorder characterized by Aβ accumulation in a subject. In some embodiments, timing of the priming of the response In some embodiments, timing of the priming of the response may be varied. In some embodiments, agents may be added to enhance a response.

Upon improvement of a patient's condition, a maintenance dose of a compound, composition or combination of this invention may be administered, if necessary. Subsequently, the dosage or frequency. of administration, or both, may be reduced, as a function of the symptoms, to a level at which the improved condition is retained. Patients may, however, require intermittent treatment on a long-term basis upon any recurrence of disease symptoms.

The compositions may be in the form of a sterile injectable preparation, for example, as a sterile injectable aqueous or oleaginous suspension. This suspension may be formulated according to techniques known in the art using suitable dispersing or wetting agents (such as, for example, Tween 80) and suspending agents. The sterile injectable preparation may also be a sterile injectable solution or suspension in a non-toxic parenterally acceptable diluent or solvent, for example, as a solution in 1,3-butanediol. Among the acceptable vehicles and solvents that may be employed are mannitol, water, Ringer's solution and isotonic sodium chloride solution. In addition, sterile, fixed oils are conventionally employed as a solvent or suspending medium. For this purpose, any bland fixed oil may be employed including synthetic mono- or diglycerides. Fatty acids, such as oleic acid and its glyceride derivatives are useful in the preparation of injectables, as are natural pharmaceutically-acceptable oils, such as olive oil or castor oil, especially in their polyoxyethylated versions. These oil solutions or suspensions may also contain a long-chain alcohol diluent or dispersant, or carboxymethyl cellulose or similar dispersing agents which are commonly used in the formulation of pharmaceutically acceptable dosage forms such as emulsions and or suspensions. Other commonly used surfactants such as Tweens or Spans and/or other similar emulsifying agents or bioavailability enhancers which are commonly used in the manufacture of pharmaceutically acceptable solid, liquid, or other dosage forms may also be used for the purposes of formulation.

The pharmaceutical compositions of this invention may be orally administered in any orally acceptable dosage form including, but not limited to, capsules, tablets, emulsions and aqueous suspensions, dispersions and solutions. In the case of tablets for oral use, carriers that are commonly used include lactose and corn starch. Lubricating agents, such as magnesium stearate, are also typically added. For oral administration in a capsule form, useful diluents include lactose and dried corn starch. When aqueous suspensions and/or emulsions are administered orally, the active ingredient may be suspended or dissolved in an oily phase is combined with emulsifying and/or suspending agents. If desired, certain sweetening and/or flavoring and/or coloring agents may be added.

The pharmaceutical compositions of this invention may also be administered in the form of suppositories for rectal administration. These compositions can be prepared by mixing a compound of this invention with a suitable non-irritating excipient which is solid at room temperature but liquid at the rectal temperature and therefore will melt in the rectum to release the active components. Such materials include, but are not limited to, cocoa butter, beeswax and polyethylene glycols.

The pharmaceutical compositions of this invention may be administered mucosally, such as by nasal aerosol or inhalation. Such compositions are prepared according to techniques well-known in the art of pharmaceutical formulation and may be prepared as solutions in saline, employing benzyl alcohol or other suitable preservatives, absorption promoters to enhance bioavailability, fluorocarbons, and/or other solubilizing or dispersing agents known in the art.

The compositions described herein can be used to treat disorders related to amyloid fibril formation (e.g., an amyloid fibril disorder). An amyloid fibril disorder includes diseases associated with the accumulation of amyloid, which can either be restricted to one organ, “localized amyloidosis”, or spread to several organs, “systemic amyloidosis.” Secondary amyloidosis can be associated with chronic infection (such as tuberculosis) or chronic inflammation (such as rheumatoid arthritis), including a familial form of secondary amyloidosis which is also seen in Familial Mediterranean Fever (FMF) and other types of systemic amyloidosis found in long-term hemodialysis patients. Some examples of disorders related to amyloid fibril formation include the following: Alzheimer's Disease, Down's Syndrome, Dutch Type Hereditary Cerebral Hemorrhage Amyloidosis, Reactive Amyloidosis, Familial Amyloid Nephropathy with Urticaria and Deafness, Muckle-Wells Syndrome, Idiopathic Myeloma; Macroglobulinemia-Associated Myeloma, Familial Amyloid Polyneuropathy, Familial Amyloid Cardiomyopathy, Isolated Cardiac Amyloid, Systemic Senile Amyloidosis, Adult Onset Diabetes, Insulinoma, Isolated Atrial Amyloid, Medullary Carcinoma of the Thyroid, Familial Amyloidosis, Hereditary Cerebral Hemorrhage With Amyloidosis, Familial Amyloidotic Polyneuropathy, Scrapie, Creutzfeldt-Jacob Disease, Gerstmann Straussler-Scheinker Syndrome, Bovine Spongiform Encephalitis, a Prion-mediated disease, and Huntington's Disease.

The term “MHC Class II molecule” as used herein, indicates an antigen-presenting molecule found primarily on dendritic cells, the best antigen presenting cells, and also on macrophages and B lymphocytes.

The term “HLA antigen” as used herein, indicates an MHC class II molecule in human.

The term “PBMC” as used herein, refers to peripheral blood mononuclear cells.

In one embodiment, “preventing, or treating” as used herein, indicates any one or more of the following: delaying the onset of symptoms, reducing the severity of symptoms, reducing the severity of an acute episode, reducing the number of symptoms, reducing the incidence of disease-related symptoms, reducing the latency of symptoms, ameliorating symptoms, reducing secondary symptoms, reducing secondary infections, prolonging patient survival, preventing relapse to a disease, decreasing the number or frequency of relapse episodes, increasing latency between symptomatic episodes, increasing time to sustained progression, expediting remission, inducing remission, augmenting remission, speeding recovery, or increasing efficacy of or decreasing resistance to alternative therapeutics. In one embodiment, “treating” refers to both therapeutic treatment and prophylactic or preventive measures, wherein the object is to prevent or lessen the targeted pathologic condition or disorder as described hereinabove.

In another embodiment, “symptoms” as used herein; indicates any manifestation of a disease or pathological condition as described hereinabove.

The administration mode of the compounds and compositions of the present invention, timing of administration and dosage, i.e. the treatment regimen, will depend on the type and severity of the injury, disease or disorder, and the age and condition of the subject. In one embodiment, the compounds and compositions may be administered concomitantly. In another embodiment, the compounds and compositions may be administered at time intervals of seconds, minutes, hours, days, weeks or more.

The patient's immune response can be measured using the techniques known to one in the art, such as measuring cytokine production or by measuring the presence of antibody in serum.

For example, a patient can provide a blood sample at an initial time where the immune response is determined. This initial determination can then be compared with the same patient's immune response at a second time, e.g., one week, one month, two months, three months, or six months later.

In one embodiment of this invention, clinicians skilled in the art will assess the progress of optimized treatment of a subject. In one embodiment of this invention, clinicians skilled in the art will assess the need for optimized immunization against a disease or disorder characterized by Aβ accumulation of a subject. For example, an antemortem diagnostic test for neurodegenerative disease may be used to follow the course of neurodegenerative disease in an individual and to validate the efficacy of a polypeptide of this invention or compositions comprising polypeptides of this invention, used to treat a neurodegenerative disease.

In some embodiments, the term “comprise” or grammatical forms thereof, refers to the inclusion of the indicated active agent, such as the compound of this invention, as well as inclusion of other active agents, and pharmaceutically acceptable carriers, excipients, emollients, stabilizers, etc., as are known in the pharmaceutical industry. In some embodiments, the term “consisting essentially of refers to a composition, whose only active ingredient is the indicated active ingredient, however, other compounds may be included which are for stabilizing, preserving, etc. the formulation, but are not involved directly in the therapeutic effect of the indicated active ingredient. In some embodiments, the term “consisting essentially of may refer to components, which exert a therapeutic effect via a mechanism distinct from that of the indicated active ingredient. In some embodiments, the term “consisting essentially of may refer to components, which exert a therapeutic effect and belong to a class of compounds distinct from that of the indicated active ingredient. In some embodiments, the term “consisting essentially of may refer to components, which exert a therapeutic effect and belong to a class of compounds distinct from that of the indicated active ingredient, by acting via a different mechanism of action, for example, and representing an embodiment of this invention, polypeptides comprising T cell epitopes present in a composition may be specifically combined with polypeptides comprising B cell epitopes. In some embodiments, the term “consisting essentially of may refer to components which facilitate the release of the active ingredient. In some embodiments, the term “consisting” refers to a composition, which contains the active ingredient and a pharmaceutically acceptable carrier or excipient.

In one embodiment, the present invention provides combined preparations. In one embodiment, the term “a combined preparation” defines especially a “kit of parts” in the sense that the combination partners as defined above can be dosed independently or by use of different fixed combinations with distinguished amounts of the combination partners i.e., simultaneously, concurrently, separately or sequentially. In some embodiments, the parts of the kit of parts can then, e.g., be administered simultaneously or chronologically staggered, that is at different time points and with equal or different time intervals for any part of the kit of parts. The ratio of the total amounts of the combination partners, in some embodiments, can be administered in the combined preparation. In one embodiment, the combined preparation can be varied, e.g., in order to cope with the needs of a patient subpopulation to be treated or the needs of the single patient which different needs can be due to a particular disease, severity of a disease, age, sex, or body weight as can be readily made by a person skilled in the art.

In some embodiments, the methods comprise ex vivo or in vitro culturing of the peptide with a dendritic cell and administering the dendritic cell to a subject. In some embodiments, the methods of this invention comprise ex vivo or in vitro culturing of the peptides as herein described, with a dendritic cell and T cells and administering the dendritic cells and T cells to a subject. In some embodiments, the peptide, dendritic cell, or T cell or a combination thereof is administered to the subject.

According to this aspect and in some embodiments, the dendritic cells, the T cells or combinations thereof are autologous or allogeneic with respect to the subject receiving the transferred cells.

In one embodiment, the term “dendritic cell” (DC) refers to antigen-presenting cells, which are capable of presenting antigen to T cells, in the context of MHC. In one embodiment, the dendritic cells utilized in the methods of this invention may be of any of several DC subsets, which differentiate from, in one embodiment, lymphoid or, in another embodiment, myeloid bone &lsqb;h embodiment, DC development may be stimulated via the use of granulocyte-macrophage colony-stimulating-factor (GM-CSF), or in another embodiment, interleukin (IL)-3, which may, in another embodiment, enhance DC survival.

In another embodiment, DCs for use in the methods of this invention may be generated from proliferating progenitors isolated from bone marrow, as is known in the art. In another embodiment, DCs may be isolated from CD34+ progenitors as described by Caux and Banchereau (Nature 360: 258-61 1992), or from monocytes, as describedby Romani et al, J. Exp. Med. 180: 83-93 1994 or Bender et al, J. Immunol. Methods, 196: 121-135, 1996. In another embodiment, the DCs are isolated from blood, as described for example, in O'Doherty et al, J. Exp. Med. 178: 1067-1078 1993 and Immunology 82: 487-493 1994, all methods of which are incorporated fully herewith by reference.

In one embodiment the DCs utilized m the methods of this invention may express myeloid markers, such as, for example, CD 11 c or, in another embodiment, an IL-3 receptor-a (IL-3Ra) chain (CD123). In another embodiment, the DCs may produce.type I interferons (JFNs). In one embodiment, the DCs utilized in the methods of this invention express costimulatory molecules. In another embodiment, the DCs utilized in the methods of this invention may express additional adhesion molecules, which may, in one embodiment, serve as additional costimulatory molecules, or in another embodiment, serve to target the DCs to particular sites in vivo, when delivered via the methods of this invention, as described further hereinbelow.

In one embodiment, the DCs may be obtained from in vivo sources, such as, for example, most solid tissues in the body, peripheral blood, lymph nodes, gut associated lymphoid tissue, spleen, thymus, skin, sites of immunologic lesions, e.g. synovial fluid, pancreas, cerebrospinal fluid, tumor samples, granulomatous tissue, or any other source where such cells may be obtained. In one embodiment, the dendritic cells are obtained from human sources, which may be, in another embodiment, from human fetal, neonatal, child, or adult sources. In another embodiment, the dendritic cells used in the methods of this invention may be obtained from animal sources, such as, for example, porcine or simian, or any other animal of interest. In another embodiment, dendritic cells used in the methods of this invention may be obtained from subjects that are normal, or in another embodiment, diseased, or in another embodiment, susceptible to a disease of interest.

Dendritic cell separation may be accomplished, in another embodiment, via any separation methods as will be appreciated by one skilled in the art, and as described in part, herein.

In one embodiment, positive and/or negative affinity based selections are conducted. In one embodiment, positive selection is based on CD86 expression, and negative selection is based on GR1 expression.

In another embodiment, the dendritic cells used in the methods of this invention may be generated in vitro by culturing monocytes in presence of GM-CSF and IL-4.

In one embodiment, the dendritic cells used in the methods of this invention may express CD83, an endocytic receptor to increase uptake of the antigen such as DEC-205/CD205 in one embodiment, or DC-LAMP. (CD208) cell surface markers, or,′ in another embodiment, varying levels of the antigen presenting MHC class I and II products, or in another embodiment, accessory is (adhesion and co-stimulatory) molecules including CD4O, CDS4, CD58 or CD86, or any combination thereof. In another embodiment, the dendritic cells may express varying levels of CDI 15, CDI4 or CD68.

In one embodiment, mature dendritic cells are obtained by the methods of this invention.

In one embodiment, the term “mature dendritic cells” refers to a population of dendritic cells with diminished CD1 15, CD14 or CD68 expression, or in another embodiment, a population of cells with enhanced p55, CD4O, CD83, CD8O or CD86 expression, or a combination thereof. In another embodiment, mature dendritic cells obtained by the methods of this invention are characterized by 80hih expression, CD831h1 expression, expression, increased MHC 25. class II expression, increased IL-12 production or a combination thereof.

In one embodiment, the maturation status of the dendritic cell may be confirmed, for example, by detecting either one or more of 1) an increase expression of one or more of p55, CD83, CD4O or CD86 antigens; 2) loss of CD1 15, CD 14, CD32 or CD68 antigen; by methods well known in the art, such as, for example, imniunohistochemistry, FACS analysis, and others.

In one embodiment, the antigen, or in another embodiment, the immune complex is delivered to dendritic cells in vivo, and in another embodiment, in the steady state. Antigen accomplished, in one embodiment, as d fh°nlet al. (2002) Journal of Experimental Medicine 196: 1627-1638; Manavalan et al. (2003) Transpl Immunol. 11: 245-58).

In another embodiment, the dendritic cell is contacted with the antigen, or in another embodiment, immune complex (IC) in vitro.

Methods for priming dendritic cells with antigen are well known to one skilled in the art, and may be effected, as described for example Hsu et al., Nature Med. 2:52-58 (1996); or Steinman et al. International application PCT/LTS93/03 141.

In one embodiment, the methods for obtaining mature dendritic cells include upregulation of costimulatory molecules on the dendritic cells, including the 137 and CD4O family of proteins.

In one embodiment, such upregulation provides for enhanced stimulation of T cell proliferation and activation, and in another embodiment, prevents T cell anergy. In another embodiment, a mature dendritic cell obtained by the methods of this invention is to be considered as part of this invention.

In one embodiment, the peptide or peptides as described herein is contacted with antigen presenting cells, which in one embodiment, are dendritic cells, prior to contact of the dendritic cells with T cells, or in one embodiment, contact of the APC's with the antigen may precede, coincide or follow contacting the APCs with the agents of this invention, as described hereinabove.

In one embodiment, soluble peptides are used at a concentration of between 10 pM to about 10 p.M. In one embodiment, 30-100 ng ml′ is used. The APCs are, in one embodiment, contacted with the antigen for a sufficient time to allow for uptake and presentation, prior to, or in another embodiment. concurrent with contact with T cells. In another embodiment, the peptide is administered to the subject, and, in another embodiment, is targeted to the APC, wherein uptake occurs in vivo, for methods as described hereinbelow.

Peptide uptake and processing, in one embodiment, can occur within 24 hours, or in another embodiment, longer periods of time may be necessary, such as, for example, up to and including 4 days or, in another embodiment, shorter periods of time may be necessary, such as, for example, about 1-2 hour periods.

The enhanced immune response obtained via the methods of this invention involves T lymphocytes. The term “T lymphocytes” or “T cells” are synonymous, and refer to a subset of lymphocytes which participate in the generation of immune responses.

In one embodiment, the T cells of this invention may be obtained from in vivo sources, such as, for example, peripheral blood, leukopheresis blood product, apheresis blood product, peripheral lymph nodes, gut associated lymphoid tissue, spleen, thymus, cord blood, mesenteric lymph nodes, liver, sites of immunologic lesions. e.g. synovial fluid, pancreas, cerebrospinal fluid, tumor samples, granulomatous tissue, or any other source where such cells may be obtained. In one embodiment, the T cells are obtained from human sources, which may be, in another embodiment, from human fetal, neonatal, child, or adult sources. In another embodiment, the T cells of this invention may be obtained from animal sources, such as, for example, porcine or simian, or any other animal of interest. In another embodiment, the T cells of this invention may be obtained from subjects that are normal, or in another embodiment, diseased, or in another embodiment, susceptible to a disease of interest.

In one embodiment, the T cells and/or dendritic cells, as described are isolated from tissue, and, in another embodiment, an appropriate solution may be used for dispersion or suspension, toward this end. In another embodiment. T cells and/or dendritic cells may be cultured in solution, in some embodiments, where the solution contains a peptide or peptides as herein described.

Such a solution may be, in another embodiment, a balanced salt solution, such as normal saline, PBS, or Hank's balanced salt solution, or others, each of which represents another embodiment of this invention. The solution may be supplemented, in other embodiment, with fetal calf serum, bovine serum albumin (BSA), normal goat serum, or other supplements, for example, as described in WO 2006/073921.PCT/US2005/047016 which, in other embodiments, may be supplied in conjunction with an acceptable buffer.

The buffer may be, in other embodiments, HEPES, phosphate buffers, lactate buffers, or the like, as will be known to one skilled in the art.

In another embodiment, the solution in which the T cells or dendritic cells may be placed is in medium is which is serum-free, which may be. in another embodiment, commercially available, such as, for example, animal protein-free base media such as X-VIVO 10or X-VIVO I 5′ (BioWhittaker, Walkersville, Md.), Hematopoietic Stem Cell-SFM media (GibcoBRL, Grand Island, N.Y.) or any formulation which promotes or sustains cell viability. Serum-free media used, may, in another embodiment, be as those described in the following patent documents: WO 95/00632; U.S. Pat. No. 5,405,772; PCT US94/09622. The serum-free base medium may, in another embodiment, contain clinical grade bovine serum atbumin, which may be, in another embodiment, at a concentration of about 0.5-5%, or, in another embodiment, about 1.0% (w/v) . Clinical grade albumin derived from human serum, such as Buminate® (Baxter Hyland, Glendale, Calif.), may be used, in another embodiment.

In another embodiment, the T cells and/or dendritic cells may be separated via affinity-based separation methods. Techniques for affinity separation may include, in other embodiments, magnetic separation, using antibody-coated magnetic beads, affinity chromatography, cytotoxic agents joined to a monoclonal antibody Or use in conjunction with a monoclonal antibody, for example, complement and cytotoxins, and “panning” with an antibody attached to a solid matrix, such as a plate, or any other convenient technique. In other embodiment, separation techniques may also include the use of fluorescence activated cell sorters, which can have varying degrees of sophistication, such as multiple color channels, low angle and obtuse light scattering detecting channels, impedance channels, etc. It is to be understood that any technique, which enables separation of the T cells or dendritic cells from a mixed source of cells, may be employed and is to be considered as part of this invention.

In another embodiment, the affinity reagents employed in the separation methods may be specific receptors or ligands for the cell surface molecules indicated hereinabove. In other embodiments, for example for T cell separations, peptide-MHC antigen and T cell receptor pairs may be used; peptide ligands and receptor; effector and receptor molecules, or others. Antibodies against T cells, which are monoclonal or polyclonal, may be used and may be produced by transgenic animals, immunized animals, immortalized human or animal B-cells, cells transfected with DNA vectors encoding the antibody or T cell receptor, etc. The details of the preparation of antibodies and their suitability for use as specific binding members are well known to those skilled in the art.

In another embodiment, any of the antibodies utilized herein may be conjugated to a label, which may, in another embodiment, be used for separation, or in another embodiment, for visualization of the target to which the antibody is bound. Labels may include, in other embodiments, magnetic beads, which allow for direct separation, biotin, which may be removed with avidin or streptavidin bound to, for example, a support, fluorochromes, which may be used with a fluorescence activated cell sorter, or the like, to allow for ease of separation, and others, as is well known in the art. Fluoroebromes may include, in one embodiment, phycobiliproteins, such as, for example, phycoerythrin, allophycocyanins, fluorescein, Texas red, or combinations thereof.

In another embodiment, the peptides of this invention are contacted with an antigen presenting cell, in vivo, or ex-vivo, and the agent is labeled with a detectable marker, such that, in one embodiment, homing, or in another embodiment, persistence of the labeled APC may be followed as a function of time.

In one embodiment, the staining intensity of the cells can be monitored by flow cytometry, where lasers detect the quantitative levels of fluorochrome (which is proportional to the amount of cell surface antigen bound by the antibodies). Flow cytometry, or FACS, can also be used, in another embodiment, to separate cell populations based on the intensity of antibody staining, as well as other parameters such as cell size and light scatter.

In another embodiment, the culture containing the T cells and/or APCs of this invention may contain cytokines or growth factors to which the cells are responsive. In one embodiment, the cytokines or growth factors promote survival, growth, function, or a combination thereof of the T and/or dendritic cells. Cytokines and growth factors may include, in other embodiment, polypeptides and non-polypeptide factors. In one embodiment, the cytokines may comprise interleukins.

In some embodiments, the T cell populations, once contacted with the dendritic cells, according to the methods of this invention, are antigen specific. In one embodiment, the term “antigen specific” refers to a property of the population such that supply of a particular antigen, or in another embodiment, a fragment of the antigen, results, in one embodiment, in specific T cell proliferation, when presented the antigen, in the context of MHC. In another embodiment, supply of the antigen or fragment thereof, results in T cell production of interleukin 2, or in another embodiment, interferon-γ, or in another embodiment, enhanced expression of the T cell receptor (TCR) on its surface, or in another embodiment, T cell function.

In one embodiment, the T cell population expresses a monoclonal T cell receptor. In another embodiment, the T cell population expresses polyclonal T cell receptors.

In one embodiment, the T cells will be of one or more specificities, and may include, in another embodiment, those that recognize a mixture of antigens derived from a single antigenic source, such as, for example, in disease, where recognition of multiple epitopes of a given antigen may be used to expand the T cells.

In one embodiment, the T cell population stimulates or enhances an immune response to a particular antigen, wherein the immune response generated is beneficial to the host.

In one embodiment, the T cell populations secrete substances, which mediate desirable effects, such as promoting the activation of other immune cells, in one embodiment, or promoting lysis in another embodiment, or apoptosis, in another embodiment. In one embodiment, the T cells of this invention mediate their effect on the immune system, without a need for direct cell contact. In one embodiment, the substances mediating these effects secreted by the T cell populations may include IL-2, interferon-γ, IL-10, IL-4, IL-17, TGFβ or a combination thereof. In another embodiment, the T cell populations may be engineered to express substances which when secreted mediate stimulatory effects on the immune system, such, as, for example, the cytokines listed hereinabove. In another embodiment, the T cell populations may be engineered to express particular adhesion molecules, or other targeting molecules, which, when the cells are introduced into the subject effect targeting of the T cell populations to a site of interest. For example, when T cell activity is desired to stimulate, or enhance an immune response at a CNS site, the T cell populations may be further engineered to express the an adhesion molecule, which has been shown to play a role in homing thereto. The cells can be engineered to express other targeting molecules, such as, for example, an antibody specific for a protein expressed at a particular site in a tissue, or, in another embodiment, expressed on a particular cell located at a site of interest, etc. Numerous methods are well known in the art for engineering the T cells and/or dendritic cells, as described herein, and may comprise the use of a vector, or naked DNA, wherein a nucleic acid coding for the targeting molecule of interest is introduced via any number of methods well described.

The invention is further described in the following examples, which do not limit the scope of the invention described in the claims.

A number of embodiments of the invention have been described. Nevertheless, it will be understood that various modifications may be made without departing from the spirit and scope of the invention. Accordingly, other embodiments are within the scope of the following claims.

### EXAMPLES

### Materials and Methods

### Antigens

Nested overlapping Aβ peptides (Aβ 1-28, Aβ 15-29, Aβ 15-34, Aβ 15-42, Aβ 16-30, Aβ 17-31, Aβ 18-32, Aβ 19-33, Aβ 20-34, Aβ 21-35, Aβ 22-36, Aβ 23-37, Aβ 24-38, Aβ 25-39, Aβ 26-40, Aβ 27-41, and Aβ 28-42) were synthesized at Biosource International (Hopkinton, Mass., USA). Aβ 1-42 used for immunizations was purchased from GenScript Corp. (Piscataway, N.J., USA). The Aβ peptides used for T-cell assays were dissolved in DMSO (2 mg/ml).

### Human Subjects

The subjects of this study were AD patients (aged 5289, n=81), healthy adults (aged 2547, n=22), and healthy older individuals (aged 5380, n=30). Patients, together with their spouses, were recruited from the Memory Disorders Unit at Brigham and Women's Hospital, Boston, under an IRB-approved human studies protocol. Diagnosis of AD was based on criteria of the National Institute of Neurological Disorders and Stroke-Alzheimer's Disease and Related Disorders Association (NINDS-ADRDA) and included use of the Mini-Mental State Examination (MMSE). Patients with mild to moderate AD and MMSE scores between 10 and 24 were selected for this study. Those with severe AD (MMSE scores below 10), or a history of severe head injury, alcoholism, major psychiatric illness, epilepsy, or learning disability, were excluded.

### Mice

Amyloid precursor protein (APP) Tg (J20 line) mice on a C57BL/6 background expressing the human mutated APPSw,Ind under the PDGF promoter (23) were kindly donated by L. Mucke. Tg mice co-expressing HLA-DR15 and a human T-cell receptor (TCR) specific for MBP 85-99 on a C57BL/6 background (24) were kindly donated by Vijay K. Kuchroo and Daniel M. Altmann. Although the latter mice carry a myelin basic protein-specific receptor, they are on a RAG wild-type background and carry an otherwise normal TCR repertoire, and they do not develop any spontaneous neuropathology. Homozygous TCR/HLA-DR15 Tg mice were bred at the animal facility of Ben-Gurion University with APP Tg mice to generate APP/HLA-DR15 double-Tg mice on a C57BL/6 background. Humanized Tg mice expressing HLA-DR0401 were obtained from Taconic (Hudson, N.Y.). C57BL/6 and SJL mice were purchased from Harlan Laboratories (Jerusalem, Israel).

### Split Multi-Well Culture System for Testing Aβ T-Cell Reactivity

PBMCs were isolated from the freshly drawn heparinized whole blood of each study participant (N=133) by Ficoll-Paque (Amersham Pharmacia Biotech, Uppsala, Sweden) gradient centrifugation, according to the manufacturer's instructions. Cells were cultured in 30 wells of 96-well round-bottom plates at 2×10⁵ cells/well in RPMI medium (containing 2.5% nonautologous serum, 4 mM L-glutamine, 10 mM HEPES, 1 mM sodium pyruvate, and 10 mM nonessential amino acids) in the presence of pure human Aβ1-42 peptide. On the 5th day of culture, 10 U/ml of recombinant human interleukin (IL)-2 was added by replacing half of the medium with IL-2-containing medium. On day 7 the medium was replaced with IL-2-free medium. On day 10, half of the cells from each well were restimulated with irradiated autologous PBMCs in the presence or absence of 5 μg/ml Aβ for 48 hours and then pulsed with [3H] thymidine for 12 hours. Cells w then harvested, their radioactivity measured (in cpm), and the stimulation index (SI; (cpm in the presence of Aβ divided by cpm in the absence of Aβ) determined. Positive wells (SI≧2.5 and Δcpm of at least 2000) obtained in the split-well assay were restimulated, 14 days after their primary stimulation, in the presence of irradiated (7000 R) autologous PBMCs (2×105 cells/well) and Aβ1-42. T-cell lines were then supplemented with IL-2 (10 U/ml) after 48 hours and every 2 days thereafter until they reached a resting state.

### Human T-Cell Line Proliferation Assays

Antigen (Ag)-specific proliferative responses were measured by culturing the contents of positive wells from the split-well assay with irradiated PBMCs (2×10⁵/well) at 37° C. in duplicate wells in 96-well U-bottom microtiter plates in a final volume of 200 μl of culture medium alone or culture medium containing serially diluted nested Aβ peptides. After 48 hours, 1 μCi of [3H]thymidine (Amersham, Arlington Heights, Ill.) was added to each well in 50 μl of culture medium. The cells were harvested 16 hours later onto fiberglass filters with a cell harvester (Packard, Filtermate 196), and [3H]thymidine incorporation was determined with a Packard Matrix 96 direct counter.

### HLA Typing

High-resolution HLA typing of PBMCs of the human subjects was carried out at the Brigham and Women's Hospital Clinical Laboratory using the PCR/RFLP method. Genomic DNA was extracted from whole blood or frozen cells. DNA was amplified using locus-specific and group-specific primers for DNA class II loci. DNA fragments were detected using agarose gel electrophoresis and subjected to restriction enzyme digestion, as recommended by the 10th International Histocompatibility Workshop (http://www.ihwg.org/components/ssopr.htm). The DNA fragments were then separated by agarose gel electrophoresis and the specific HLA-DR alleles were analyzed visually.

### Aβ Immunization of Mice

Mice were immunized by subcutaneous injection of Aβ 1-42 (100 mg) emulsified in complete Freund's Adjuvant (CFA) H37Ra (Difco, Detroit, Mich., USA) or in incomplete Freund's Adjuvant (IFA; Sigma, Israel), as indicated in Figure legends. When indicated, pertussis toxin (PTX) (List Biological Laboratories, Campbell, Calif., USA) was injected intravenously (200 ng/mouse) on day 0 and again 48 hours later. For booster immunizations Aβ1-42 was emulsified in IFA and injected subcutaneously at the intervals indicated in Figure legends.

### Proliferation Assay of Mouse-Derived T Cells

Popliteal lymph nodes or spleens of immunized mice were harvested and cultured (5×106 cells/ml) in U-shaped 96-well-plate culture dishes in DMEM containing 10% fetal calf serum, 10 mM HEPES, 1 mM sodium pyruvate, 10 mM nonessential amino acids, 1% Pen/Strep, and 50 mM b-mercaptoethanol. After 72 hours, 3[H]thymidine (1 mCi) was added. Cells were harvested after 12 hours, and their radioactivity was measured.

### Cytokine ELISA

Lymph node-derived or spleen-derived cells were cultured (10×10⁶ cells/ml) as described above. IL-2 and IL-4 were measured in the supernatants after 24 hours, interferon (IFN)-γ after 48 hours, and IL-17 after 72 hours, in each case by means of a sandwich ELISA according to the manufacturer's instructions (Biolegend, San Diego, Calif., USA).

### ELISA of Antibodies

Serum samples were subjected to ELISA using the Mouse Immunoglobulin Isotype Panel kit (Southern Biotech, Birmingham, Alabama, USA) according to the manufacturer's instructions. Plates were coated with peptides or purified ovalbumin (OVA) (3 mg/ml) and sera were diluted to 1:5000.

### Immunohistochemistry

Mice were killed with an overdose of isoflurane, and their brains were rapidly removed and placed in moulds containing an OCT compound (Tissue-Tek, Torrance, Calif., USA). The tissues were frozen in isopentane, cooled in liquid nitrogen, and stored at −80° C. Sagittal sections (12 μm) were taken throughout the hippocampus and fixed in ice-cold methanol for 2 minutes, then in 4% formaldehyde for 4 minutes, and then washed with double-distilled water and phosphate-buffered saline (PBS)/Tween (0.05%). Prior to staining, primary antibody diluting buffer (Biomeda Corp., Foster City, Calif., USA) was used to block nonspecific binding. Anti-CD11b (1:25) was purchased from Serotec, Raleigh, N.C., USA, and anti-CD4 (1:50) from Biolegend, San Diego, Calif., USA. Rabbit anti-human Aβ antibodies (1:500) were generated at the animal facility of Ben-Gurion University and examined for specificity by ELISA and immunohistochemistry. TO-PRO 3 (Molecular Probes, Invitrogen, USA) was used at a dilution of 1:3000, and all subsequent secondary antibodies were conjugated to Alexa-488, Alexa-546, or Alexa-647 (Invitrogen, Carlsbad, Calif., USA) diluted 1:500. Sections were examined under an Olympus Fluoview FV1000 confocal laser scanning microscope.

### Confocal Imaging Analysis

Brains were quantitatively analyzed for Aβ plaques and CD11b+cells using four sections (12 μm thick) per hemisphere for accurate representation of the hippocampus area. Sections were stained for Aβ and CD11b and examined with the confocal microscope. Fluorescence intensity was first obtained in sections from control mice (immunized with adjuvant only), and identical laser-scanning parameters were then used for the entire experiment. Using Volocity 3D image analysis software (Improvision, Waltham, Mass., USA), we set an intensity threshold to mark only those areas showing significant staining. The average of the summed fluorescence area per brain section was calculated for each of the analyzed groups.

### Statistical Analysis

The frequency of each HLA-DRB1 allele in the study group was calculated by dividing the number of subjects bearing a specific allele by the total number of subjects in the study and then multiplying by 100. Data of the various experiments were analyzed by a Student's t-test, two-tailed (Graph Pad Prism 5.0).

### EXAMPLE 1

### Epitope Specificity and HLA-DR Restriction of Aβ-Reactive T-Cell Lines

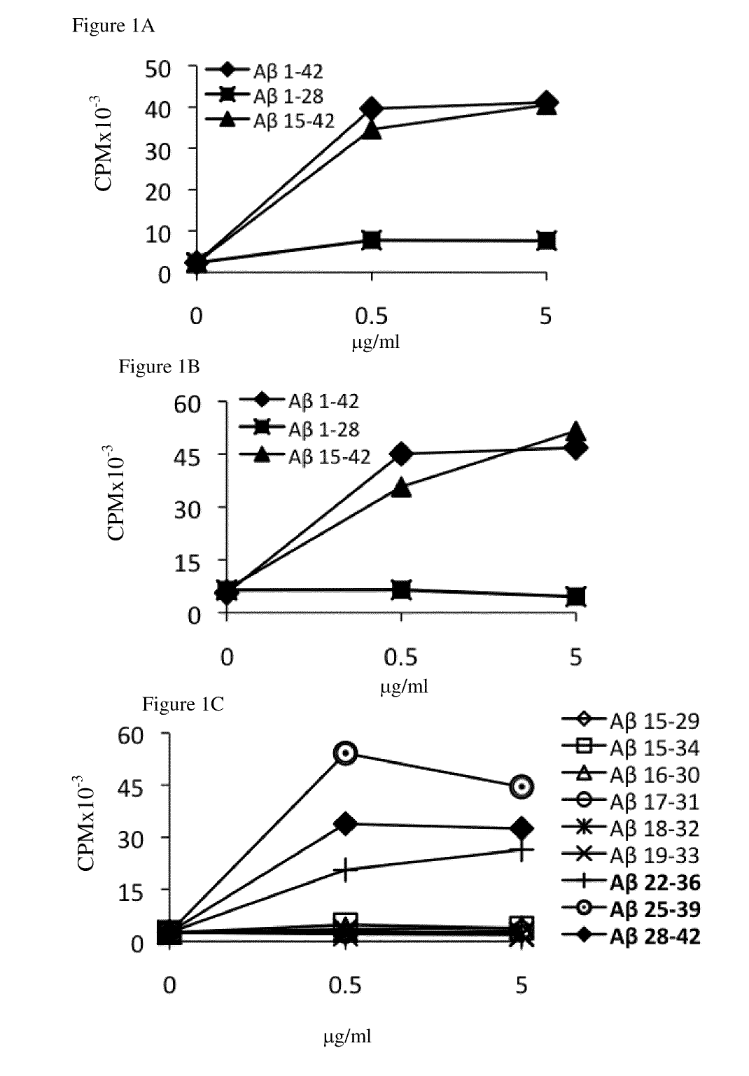

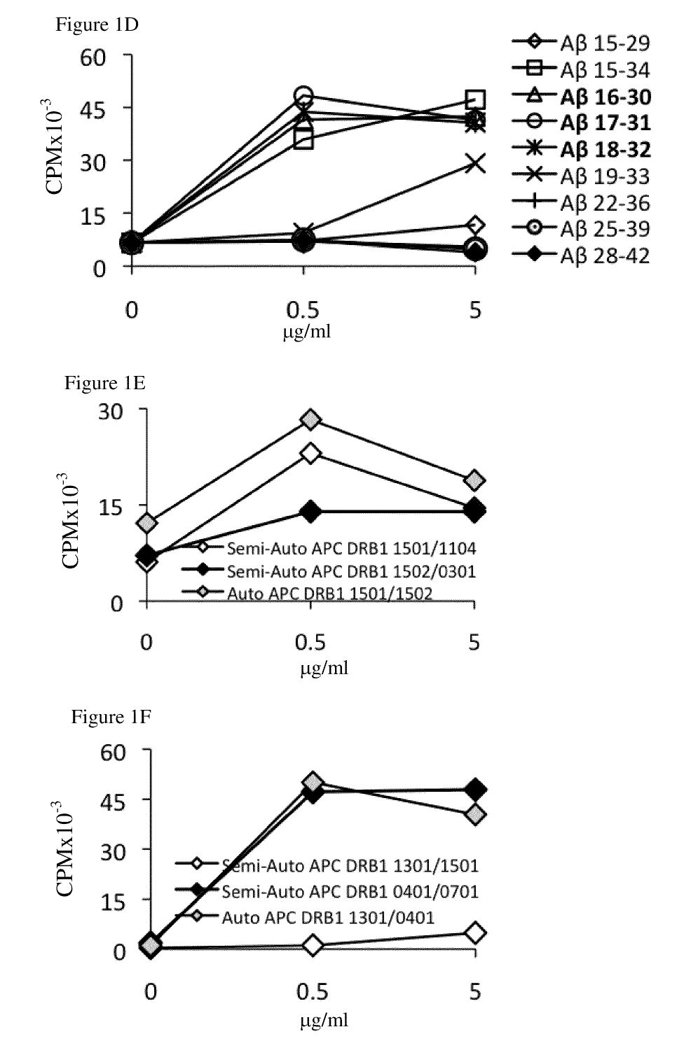

To identify the HLA-DR alleles that play a role in Aβ immunogenicity, Aβ-reactive T-cell lines were generated from the PBMCs of human subjects as described in Materials and Methods. These T cells were then restimulated with autologous or semi-autologous PBMCs (nonautologous PBMCs bearing one of the autologous DRB1 alleles) to determine their epitope specificity and identify their dominant epitope-presenting HLA-DR alleles. Overall, the PBMCs of 89 of the 133 individuals analyzed in this study yielded positive Aβ-reactive T-cell responses in the split-well assay as described in Materials and Methods and our previous publication ((2003). J Clin Invest 112:415-422). From these 89 PBMC samples, 39 Aβ-reactive T-cell lines were successfully generated and were analyzed for epitope specificity and HLA-DR allele restriction, as shown in FIG. 1. Table 2 lists the HLA-DR alleles whose expression by PBMCs was essential for proliferation of the Aβ-specific T-cell lines. The table also records the frequency of the HLA-DR alleles in our study population and lists the Aβ peptides harboring the dominant T-cell epitopes.

FIG. 1 depicts the Aβ-epitope specificity of two T-cell lines (“line 1” and “line 2”) generated from the PBMCs of two individuals bearing different HLA-DR alleles. As indicated by [3H] thymidine incorporation, proliferation of both T-cell lines, was triggered by their stimulation with autologous PBMCs and increasing concentrations of Aβ1-42 and Aβ15-42 (FIGS. 1A and B; SI values are recorded in Figure legends). Stimulation with Aβ1-28 yielded only mild T-cell proliferation of line 1 but not of line 2 (FIGS. 1A and B).

To determine the fine epitope specificity of these T-cell lines, a T-cell proliferation assay was conducted using nested (15-amino-acid long) peptides of Aβ15-42 (FIGS. 1C and D). Whereas T-cell line 1 was specifically stimulated by the C-terminal peptide Aβ25-39 and—though to a lesser extent—by the flanking peptides Aβ22-36 and Aβ28-42 (FIG. 1C, SI values are recorded in Figure legends), T-cell line 2 was stimulated by a middle Aβ fraction, Aβ15-34, and within this fraction specifically by Aβ16-30, Aβ17-31, and Aβ18-32 (FIG. 1D). Substantially lower T-cell proliferation was stimulated by Aβ19-33, suggesting that the T-cell epitope is located between residues 18 and 32 (FIG. 1D).

To identify the specific DRB1 alleles presenting the Aβ peptides, both T-cell lines were stimulated with autologous or semi-autologous PBMCs and Aβ1-42. FIG. 1E shows that T-cell line 1 underwent proliferation when stimulated with autologous PBMCs bearing the DRB1 1501/1502 alleles or with semi-autologous PBMCs bearing the DRB1 1501/1104 alleles. Cells of line 2 proliferated when stimulated with autologous PBMCs bearing the DRB1 1301/0401 alleles or the semi-autologous PBMCs bearing the DRB1 0401/0701 alleles (FIG. 1F). Only slight proliferation was observed when T-cell lines 1 and 2 were stimulated with semi-autologous PBMCs bearing the 1502/0301 and 1301/1501 HLA-DR alleles, respectively. It thus seems reasonable to suggest that the DRB1 alleles 1501 and 0401 are the dominant Aβ-presenting alleles in T-cell lines 1 and 2, and that the T-cell epitopes they present are located between Aβ residues 25 and 42 and between 18 and 32, respectively.

As summarized in Table 2, presentation of peptides between Aβ residues 25 and 42 was associated with expression of the DRB1 * 1501 allele, which was also the one that occurred most frequently (30%) in the study group. Presentations of peptides between residues 15 and 34 of Aβ was associated with expression of the less frequent but still relatively abundant alleles of the DR4 (12.5%), DR1 (11%), DR13 (8%) and DR10 (3%) haplotypes (Table 1). Expression of the DRB1*0301 and DRB1*1502 alleles, which occurred in 18% and 6% of our study group, respectively, was associated with presentation of T-cell epitopes over a wide range of Aβ residues (between 15 and 42; Table 1), and further analysis is needed for more precise definition of their specificity. The legend to the table records the frequencies of HLA-DR alleles expressed in subjects whose T cells displayed significant Aβ reactivity in the split-well assay but whose T-cell epitopes and allele restriction have not yet been analyzed. Notably, when used as semi-autologous antigen-presenting cells, PBMCs bearing the DRB1 alleles 1101 (9%), 0801 (3.8%), and 0102 (3.8%) did not stimulate proliferation of Aβ-reactive T cells.

Table 2 presents the frequency and Aβ epitope specificity of HLA-DR alleles in human subjects.

TABLE 2

| DR | | Frequency of T-cell- | T-cell-stimulatory Aβ |
|---|---|---|---|
| haplotype | DRB1 alleles | stimulatory alleles (%) | peptides |
| DR15 | DRB1*1501 | 30% | 25-42 |
| | DRB1*1502 | 6% | 15-42 |
| DR3 | DRB1*0301 | 18% | 15-42 |
| DR4 | DRB1*0404 | 5% | 18-32 |
| | DRB1*0401 | 7.5% | 18-32 |
| DR1 | DRB1*0101 | 11% | 15-34 |
| DR13 | DRB1*1301 | 8% | 17-31 |
| DR10 | DRB1*1001 | 3% | 15-29 |

Blood samples drawn from 133 human subjects were analyzed for Aβ-reactive T cells and HLA-DR alleles, as described in Materials and Methods. Aβ-reactive T-cell lines were generated and their restriction to HLA-DR haplotypes was analyzed by the use of autologous and semi-autologous PBMCs as antigen-presenting cells. Calculated frequencies of the alleles found to directly stimulate Aβ-reactive T cells in our subjects are shown. Also recorded are the Aβ peptides (were obtained using nested overlapping peptides of Aβ that stimulated the proliferation of such T cells. HLA-DR alleles of human subjects where Aβ-induced T-cell proliferation was observed but was not further analyzed for epitope specificity are as follows (frequency of presence in the study population is shown in brackets): DRB1 1104 (11%), DRB 1 0402 (6%), DRB1 1302 (3%), DRB1 0403 (3%), DRB1 1102 (1.5%), DRB1 0103 (1.5%), DRB1 0407 (1.5%), DRB1 0302 0.8%), DRB1 1404 (0.8%).

### EXAMPLE 2

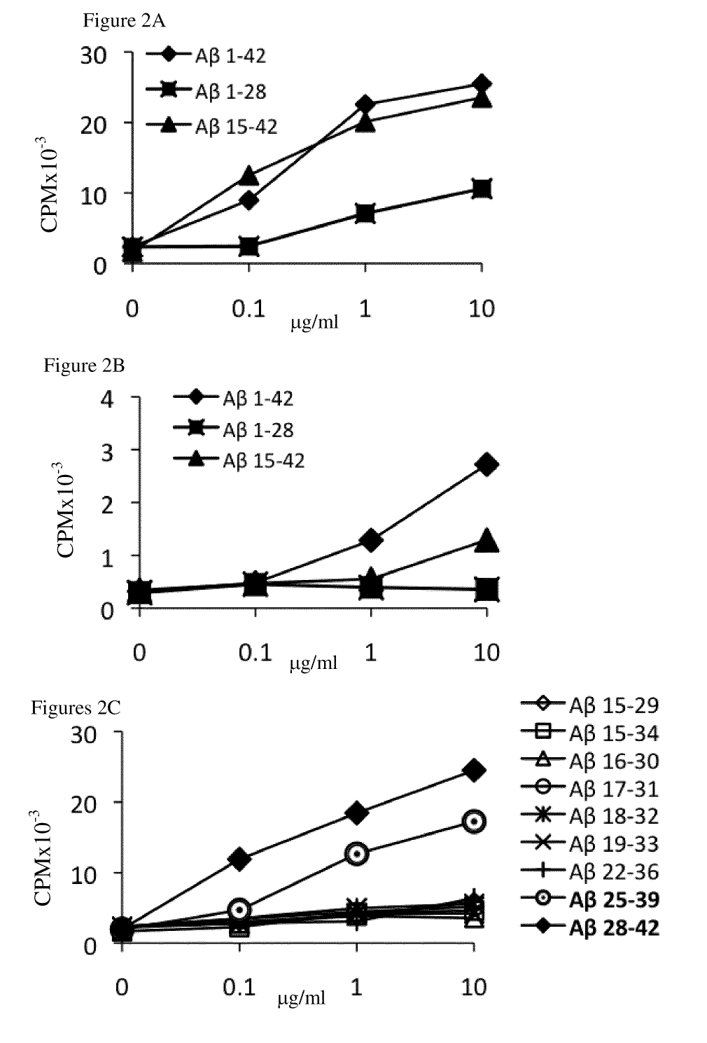

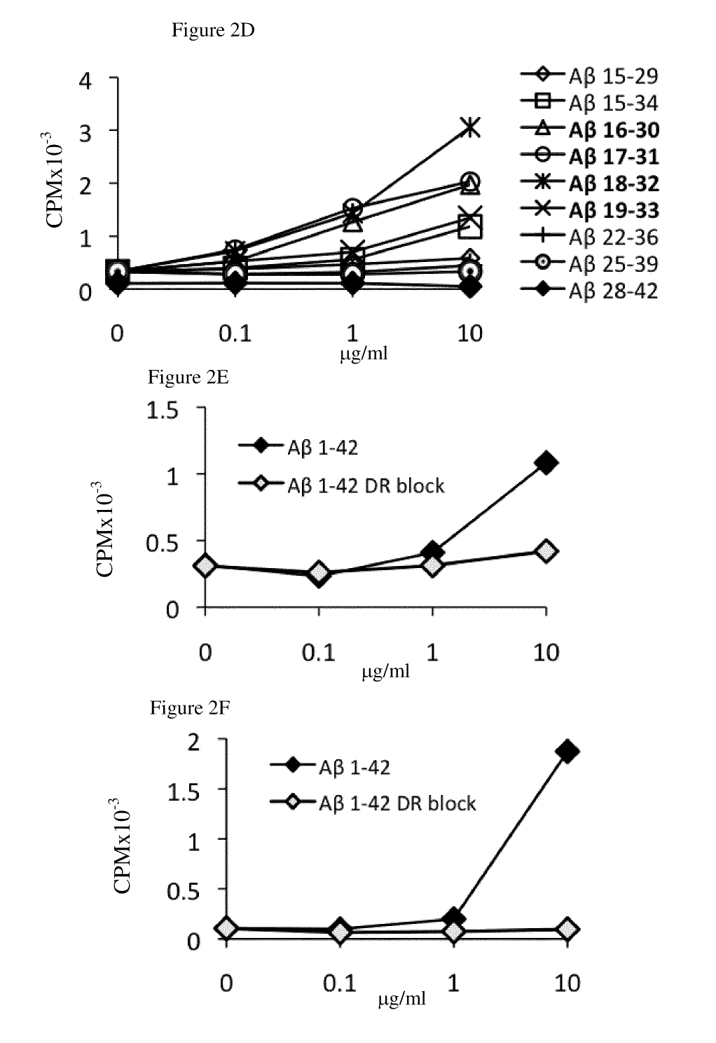

AβT-cell epitopes in HLA-DR15 and HLA-DR4 humanized mice To confirm the above data on Aβ epitope specificity obtained for human Aβ-reactive T-cell lines and their restriction to specific HLA-DR alleles humanized mouse models expressing the DRB1*1501 (DR15) and DRB1*0401 (DR4) genes of the DR15 and DR4 haplotypes, respectively were used. DR15 and DR4 Tg mice were immunized with Aβ1-42 emulsified in CFA. To increase the frequency of Aβ-reactive T cells in the spleen, and hence the proliferative response to the various Aβ peptides, mice were also given a subsequent booster injection of Aβ1-42 emulsified in IFA. The mice were killed 18 days later and their spleens were analyzed for Aβ-specific T-cell proliferation as described in Materials and Methods. For this analysis we first used increasing amounts of Aβ1-42 peptide, the C-terminal fragment Aβ15-42, and the N-terminal fragment Aβ1-28. In mice bearing the DRB1*1501 allele, the first two promoted strong T-cell proliferation, whereas only a mild T-cell response was induced by Aβ1-28 (FIG. 2A; all SI values are recorded in the Figure legends). As with DR15 Tg mice, T-cell proliferation in mice bearing the DR4 allele was induced by the Aβ peptides 1-42 and Aβ15-42, but not by the N-terminal fragment Aβ1-28 (FIG. 2B). Fine epitope analysis, carried out with the nested peptide cassette of Aβ15-42, revealed that the dominant T-cell epitopes were mapped to Aβ residues 25-42 (FIG. 2C) and 16-32 (FIG. 2D) in DR15 and DR4 mice, respectively. In both DR15-derived (FIG. 2E) and DR4-derived cultures (FIG. 2F), T-cell proliferation induced by Aβ1-42 was blocked in the presence of DR antibodies. The observed localization of the T-cell epitopes at Aβ residues 25-42 and 16-32 further confirmed the specificity of the epitopes determined in the T-cell lines of our human subjects bearing the DRB1*1501 and DRB1*0401 alleles, respectively.).

### EXAMPLE 3

### T-Cell and B-Cell Responses Elicited in Aβ-Immunized APP/DR15 Tg Mice

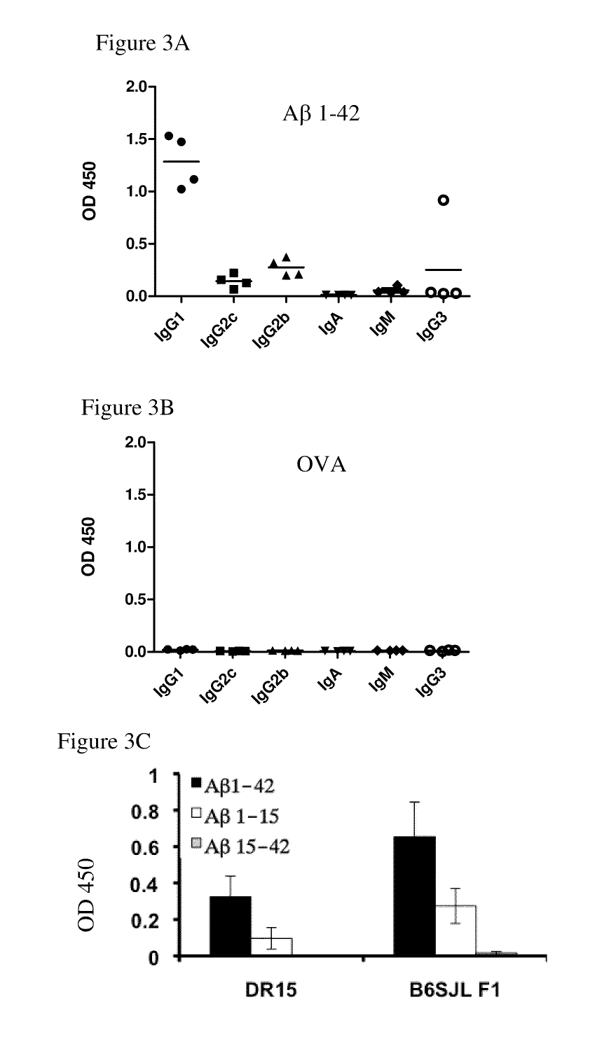

To identify the isotypes of Aβ antibodies produced in DR15 Tg mice, 2-month-old mice were vaccinated with Aβ1-42 emulsified in CFA, followed by two booster injections of Aβ1-42 emulsified in WA at 2-week intervals. The different isotypes of Aβ-specific antibodies produced in serum samples 2 weeks after the last immunization were measured as described in Materials and Methods. As shown in FIG. 3A, Aβ immunization resulted in the production mainly of IgG1 specific antibodies, with significantly less production of IgG2c, IgG2b, IgG3, and IgM. No specific binding to OVA was observed for the various antibody isotypes (FIG. 3A). As reported for other mouse strains (20, 25, 26), sera bound to Aβ1-42-coated wells also bound to wells coated with Aβ1-15 but not with Aβ15-42 (FIG. 3B).

### EXAMPLE 4

### Aβ]Immunization of APP/DR15 Tg Mice Promotes Clearance of Amyloid Plaques

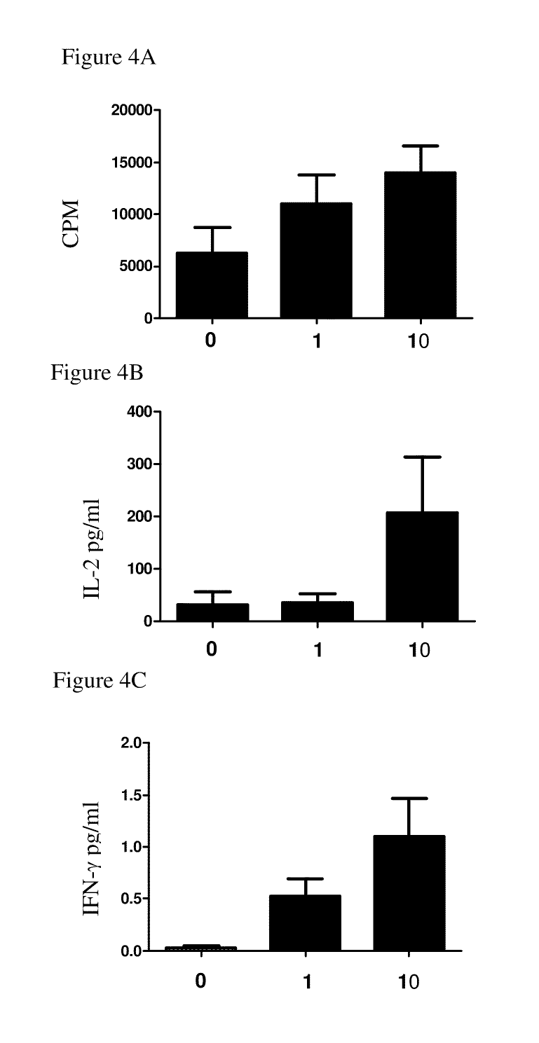

We next sought to determine the long-term effects of Aβ immunization of APP Tg mice bearing the DRB1*1501 allele on the generated immune response and on Aβ deposition in the brain. APP/DR15 Tg mice aged 7 months were immunized with Aβ1-42 emulsified in CFA and injected intravenously with PTX as described in Materials and Methods. The mice were immunized 3.5 and 7 weeks later, with Aβ1-42 emulsified in IFA. At 9.5 months of age (3.5 weeks after the last immunization) the mice were killed and their splenocytes were analyzed for Aβ-specific T- and B-cell responses. Splenocyte cultures assayed 72 hours after Aβ stimulation revealed dose-dependent T-cell proliferation (FIG. 4A). The growth-factor cytokines IL-2 and IL-4 were assayed 24 hours after T-cell stimulation with Aβ1-42, and the effector-function cytokine IFN-γ was assayed 24 hours later. The results showed specific and dose-dependent secretion of IL-2 (FIG. 4B) but not of IL-4 (data not shown). Notably, while IL-2 concentration at 20 μg/ml Aβ was about half of its concentration assayed in lymph nodes 13 days after immunization, concentrations of IFN-β were reduced about 100 fold, to about 1 ng/ml (FIG. 4C). Thus the immune response to Aβ generated in APP/DR15 Tg mice closely resembled the pattern of the response obtained on day 13 after immunization, but was much weaker. The decline in response strength might be attributable to the older age of the immunized mice and/or to the possibility that the immunization regimen, in the long term, yields more antibody production and less T-cell activation.

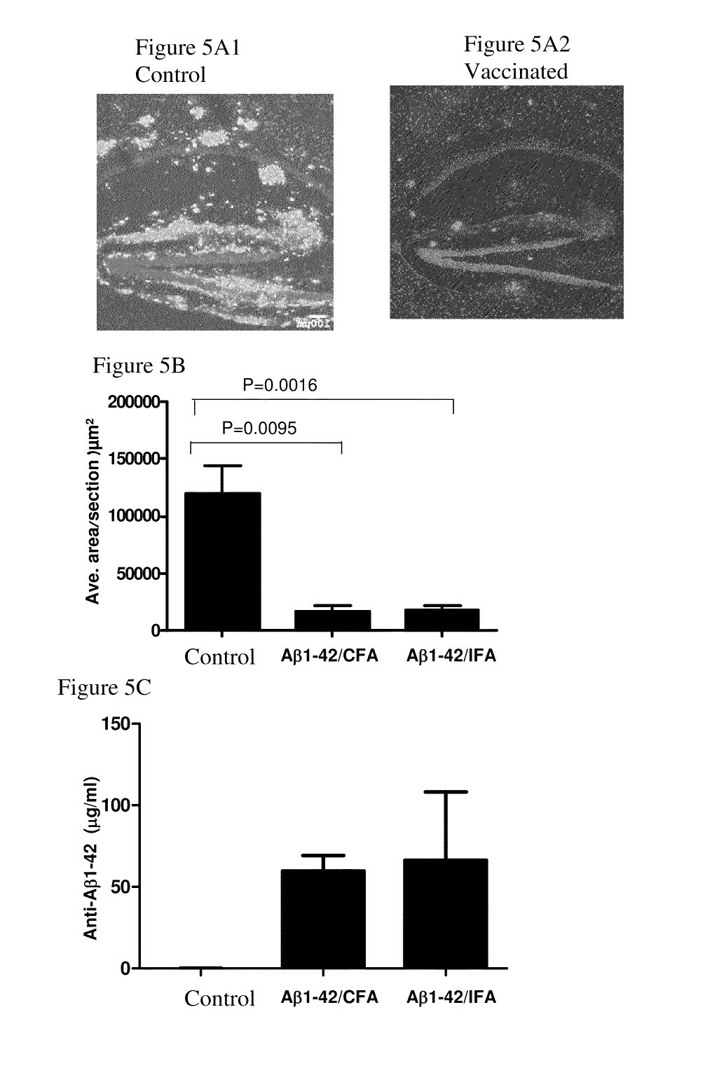

To examine the efficiency of amyloid clearance from the brains of Aβ-immunized APP/DR15 Tg mice, mice were immunized as described for FIG. 4. In addition, 7-month-old APP/DR15 Tg mice (n=4) were immunized with Aβ1-42 emulsified in IFA, and 3.5 and 7 weeks later with Aβ1-42 emulsified in IFA. Control mice were immunized with the CFA and IFA adjuvants only (n=5). At 3.5 weeks after the last immunization the mice were killed and their brains analyzed for clearance of Aβ. Aβ plaques in brain sections were immunostained and then quantified stereologically as described in Materials and Methods. Representative images of brain sections of □□ vaccinated mice show remarkably fewer Aβ deposits (green) in the hippocampal dentate gyrus area than in control mice immunized with adjuvant only (FIG. 5A). Use of the Volocity image-analysis software for quantification of Aβ demonstrates a significant reduction in □□ in the area of the hippocampal dentate gyrus in both groups of Aβ-immunized mice compared to the adjuvant-immunized control group, suggesting that the primary mechanism of clearance is via Aβ antibodies (FIG. 5B). As shown in FIG. 5C, the amounts of total IgG anti-Aβ antibodies were 59.7±9.5 and 66:3±41.9 μg/ml in Aβ/CFA and Aβ/IFA immunized mice, respectively. No specific Aβ antibodies were detected in control mice immunized with adjuvant only (FIG. 5C).

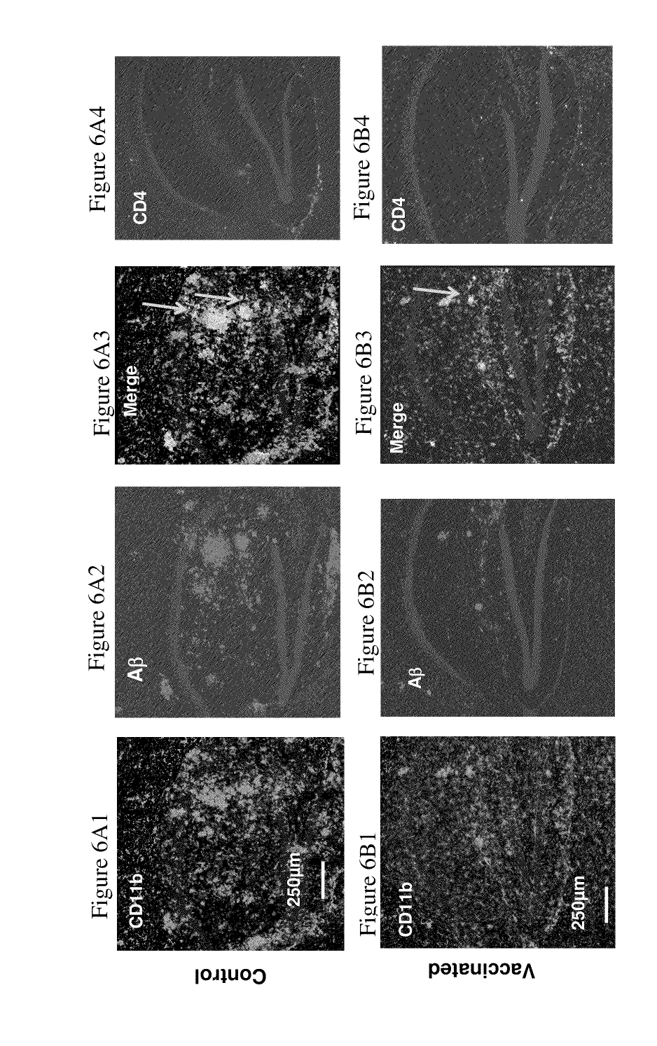

To characterize the inflammatory response associated with Aβ clearance, brain sections were immunostained with Aβ, CD11b, and CD4 antibodies. Co-staining of Aβ (red) and CD11b (green) shows co-localization between Aβ plaques and activated microglia in the hippocampi of both Aβ-immunized and control mice (FIG. 6A, see arrows at the merged panels). Importantly, the significant decrease in Aβ plaque-formation in the vaccinated group was accompanied by a proportional decrease in the amount of activated microglia in the brain (FIG. 6A). Use of the Volocity image-analysis software for quantification of Aβ demonstrated a significant reduction both in the mean area occupied by CD11b+ cells (FIG. 7B, left panel) and in their mean fluorescence intensity (FIG. 6B, right panel) in the Aβ/IFA-vaccinated mice compared to controls. A similar reduction in CD11b-immunostained cells was observed in the Aβ/CFA group (data not shown). To determine whether the clearance of Aβ in this humanized mouse model of AD was associated with infiltrating lymphocytes, we immunostained brain sections of Aβ-immunized and control mice with CD4 antibodies. FIG. 6 demonstrates only a few T cells, which in both groups of mice were detected in the meninges but not in the brain parenchymal tissues, suggesting that the lymphocyte infiltration is a nonspecific effect of the adjuvant.

The above-described Examples demonstrates for the first time that Aβ T-cell epitopes in human subjects are presented by specific HLA-DR alleles. This discovery throws new light on the nature of Aβ as an immunogen in the human population. One such HLA-DR DRB1 allele is the highly prevalent allele DRB1*1501, which presents a T-cell epitope located at the C-terminal portion of the Aβ peptide, between residues 25 and 42 of Aβ. Moreover, this T-cell epitope is highly immunogenic in humanized DR15 Tg mice, eliciting a strong Th1/Th17 T-cell response and primarily IgG1 and IgG2b humoral responses. Furthermore, prophylactic immunization of old APP Tg mice bearing the DRB1*1501 allele induced effective clearance of amyloid plaques and consequently a decrease in the activation of microglia in the brain parenchyma.

The Examples demonstrates the specific restriction of these epitopes to HLA-DR alleles. PBMCs bearing the DRB1*1501 allele (30% in our study population) stimulated Aβ-specific T-cell lines via epitopes located at Aβ residues 25-39 and 28-42. T-cell epitopes at Aβ residues 15-42, 15-34, 17-31, 18-32, and 15-29 were associated with the less prevalent DRB1 alleles 0301 (18%) and 1502 (6%), 0101 (11%), 1301 (8%), 0404 (5%) and 0401 (7.5%), and 1001 (3%), respectively. Notably, about 20% of our subjects were found to bear HLA-DR alleles such as DRB1*1101 (9%), which either did not stimulate the Aβ-reactive T-cell lines we studied or promoted only a mild response. However, because each individual expresses two HLA-DR alleles, it is possible that one of them predominates. As shown in FIG. 1, the DRB1*1502 and DRB1*1301 induced only weak T-cell proliferation when coexpressed with DRB1*1501 or DRB1*0401, respectively, but they were dominant when co-expressed with other HLA-DR alleles (Table 2).

It thus seems that Aβ-presenting alleles were expressed in the majority of the analyzed subjects, but that Aβ immunogenicity may vary substantially in magnitude and effector function, depending primarily on the type of HLA-DR genetic background and the T-cell epitope presented.

T-cell responses were further determined in humanized mice bearing HLA-DR alleles. Following Aβ1-42 immunization of humanized mice bearing the DRB1 1501 and 0401 alleles, peptides between residues 25 and 42 and between 18 and 32 were found to serve as the dominant T-cell epitopes, as observed also for T-cell lines derived from human subjects with these HLA genetic backgrounds. The overlapping peptides between residues 15 and 42 of Aβ have never been probed in this context, and the different T-cell epitopes obtained might thus reflect the presence of an additional weak T-cell epitope located at the N-terminus of Aβ or suggest that only a truncated portion of the epitope was presented to T cells. Overall, the strongest T-cell responses in the humanized mice were to peptides derived from Aβ residues 15-42, similar to our findings in human PBMCs bearing various HLA-DR alleles (Table 1).

The use of HLA-DR humanized mice makes it possible to determine both the specificity and the immunogenic characteristics of Aβ in humans, which—as observed in our APP Tg mice bearing the DRB1*1501 allele—appear to be remarkably different from those in mouse MHC-II alleles. Firstly, the dominant T-cell epitope in DR15 mice was found between residues 25 and 42. Secondly, the amounts of IFN-γ secreted as a consequence of in-vitro stimulation of DR15-derived T cells were about 5-20 times higher than those observed with T cells derived from the highly immunogenic SJL strain (data not shown). It is therefore possible that the large amounts of IFN-γ and IL-17 induced in the Aβ-reactive T cells of DR15 Tg mice reflect their pathogenic capacity upon immunization with a strong adjuvant such as CFA.

Prophylactic immunization of APP/DR15 Tg mice with Aβ/CFA followed by several boosters of Aβ/IFA evoked a high titer of Aβ antibodies, which—as indicated in numerous reports—promoted significant clearance of Aβ and also lowered the degree of microglial activation in the brain parenchyma. These types of immunization protocols yield high titers of Aβ antibodies even before there is significant deposition of Aβ takes place in the brain. It is therefore possible that the continuous clearance of soluble forms of Aβ, induced by circulating specific antibodies results in less plaque formation in the brain with age and hence fewer plaque-associated activated microglia. Alternatively, because activated microglia were found to be co-localized with almost all Aβ plaques in the brain (FIG. 6), it is possible that once a plaque is generated it can be removed more effectively if Aβ antibodies are present.

While certain features of the invention have been illustrated and described herein, many modifications, substitutions, changes, and equivalents will now occur to those of ordinary skill in the art. It is, therefore, to be understood that the appended claims are intended to cover all such modifications and changes as fall within the true spirit of the invention.

1-79. (canceled)

80. A method of optimizing treatment of a subject afflicted with a disease or disorder associated with amyloid beta accumulation or optimizing immunization against a disease characterized by amyloid beta accumulation in a subject, said method comprising:
determining expression of an HLA-DR allele in a sample derived from a subject diagnosed with the disease or disorder, said HLA-DR being indicative of treatment regimen of the subject using an amyloid beta peptide,
thereby optimizing treatment of the subject afflicted with the disease or disorder associated with amyloid beta accumulation or optimizing immunization against the disease characterized by amyloid beta accumulation in the subject.

81. The method of claim 80, wherein said HLA-DR allele is HLA-DRB1 allele.

82. The method of claim 80, wherein when
(i) said subject expresses an HLA-DRB1 1501 allele, then said treatment regimen comprises administering an amyloid beta peptide selected from the group consisting of SEQ ID NOs: 1, 2, 7, 14, 20, 21, and 27-34;
(ii) said subject expresses an HLA-DRB1 0301 allele, then said treatment regimen comprises administering an amyloid beta peptide selected from the group consisting SEQ ID NOs: 3-7;
(iii) said subject expresses an HLA-DRB1 0101 allele, then said treatment regimen comprises administering an amyloid beta peptide selected from the group consisting SEQ ID NOs: 3, 4, 5, 7, 8, 22-28;
(iv) said subject expresses an HLA-DRB1 1301 allele, then said treatment regimen comprises administering an amyloid beta peptide selected from the group consisting SEQ ID NOs: 7, 9, 10, 24-26;
(v) said subject expresses an HLA-DRB1 1502 allele, then said treatment regimen comprises administering an amyloid beta peptide selected from the group consisting SEQ ID NOs: 3-5, 7;
(vi) said subject expresses an HLA-DRB1 0404 allele, then said treatment regimen comprises administering an amyloid beta peptide selected from the group consisting SEQ ID NOs: 7, 9, 10,11, 19, 24-26;
(vii) said subject expresses an HLA-DRB1 1001 allele, then said treatment regimen comprises administering an amyloid beta peptide selected from the group consisting SEQ ID NOs: 7, 12,13, 22-24;
(viii) said subject expresses an HLA-DRB1 0401 allele, then said treatment regimen comprises administering an amyloid beta peptide selected from the group consisting SEQ ID NOs: 7, 9, 11, 13, 15, 18, 22-24. ;

83. The method of claim 80, wherein said amyloid beta peptide is administered as part of a pharmaceutical composition.

84. The method of claim 83, wherein said pharmaceutical composition comprises a neuroprotective agent, a neurotrophic factor or a combination thereof.

85. The method of claim 84, wherein said neuroprotective agent increases brain levels of interferon-γ.

86. The method of claim 83, wherein said pharmaceutical composition comprises an agent which increases brain levels of Treg cells.

87. The method of claim 83, wherein said pharmaceutical composition comprises an adjuvant.

88. The method of claim 83, wherein said pharmaceutical composition is in a form suitable for intracranial administration.

89. The method of claim 83, wherein said pharmaceutical composition is in a form suitable for intranasal administration.

90. The method of claim 80, wherein said disease or disorder is a neurodegenerative disease or disorder.

91. The method of claim 80, wherein said disease or disorder is an amyloidosis disease or disorder.

92. The method of claim 80, wherein said peptide is cultured ex vivo or in vitro with an antigen presenting cell, a T cell or a combination thereof and said antigen presenting cell, T cell or combination thereof is then administered to said subject.

93. A method of determining responsiveness of a subject afflicted with a disease or disorder associated with amyloid beta accumulation to an amyloid beta peptide-dependent treatment, comprising determining expression of an HLA-DR allele in a sample derived from a subject diagnosed with the disease or disorder, wherein expression of said HLA-DR allele being indicative to the responsiveness of the subject to the amyloid beta peptide-dependent treatment,
thereby determining the responsiveness of the subject afflicted with the disease or disorder associated with amyloid beta accumulation to the amyloid beta peptide-dependent treatment.

94. The method of claim 93, wherein
(i) when said subject expresses an HLA-DRB1 allele selected from the group consisting of 1501, 0301, 0101, 1301, 1502, 0404, 1001, 0401, 1104, 0402, 04011, 1302, 0403, 1102, 0103, 0407, 0302, and 1404, then said subject will be responsive to an amyloid beta peptide-dependent treatment regimen; and
(ii) when said subject expresses any two of the HLA-DRB1 allele selected from the group consisting of 1101, 0801, 0102, 00170, and 0701, then said subject is considered to be non-responsive to an amyloid beta peptide-dependent treatment regimen.

95. A polypeptide consisting of an amino acid sequence as set forth in SEQ ID NOs: 1, 3-6, 9, 12, 22 or 23.

96. A pharmaceutical composition comprising the polypeptide of claim 95.

97. The pharmaceutical composition of claim 96, further comprising an amino acid sequence as set forth in any one of SEQ ID NOs: 2, 7, 8, 10, 11, 13-15, 17-21, 24-34, or any combination thereof.

98. The pharmaceutical composition of claim 96, further comprising a neuroprotective agent, a neurotropic factor, or a combination thereof.

99. The pharmaceutical composition of claim 98, wherein said neuroprotective agent is an agent which increases levels of interferon-γ.

100. The pharmaceutical composition of claim 96, comprising an agent which increases levels of Treg cells.

101. The pharmaceutical composition of claim 96, comprising an adjuvant.

102. The pharmaceutical composition of claim 96, wherein said pharmaceutical composition is in a form suitable for intracranial administration.

103. The pharmaceutical composition of claim 96, wherein said composition is in a form suitable for intranasal administration.

104. A method of optimizing treatment of a subject afflicted with a disease or disorder associated with amyloid beta accumulation, comprising administering at least one polypeptide to said subject, wherein
(i) when said subject is a Caucasian subject said polypeptide has an amino acid sequence as set forth in any one of SEQ ID NOs: 1-7, 14, 20-34, or a combination thereof;
(ii) when said subject is an Asian or an Arab subject then said polypeptide has an amino acid sequence as set forth in any one of SEQ ID NOs: 1-7, 9, 11-15, 18, 20-24, 27-34, or a combination thereof;
(iii) when said subject is an African subject, then said polypeptide has an amino acid sequence as set forth in any one of SEQ ID NOs: 3-7, 9, 10, 12, 13, 22-24, or a combination thereof;
(iv) when said subject is an African American subject, then said African American subject, wherein said polypeptide has an amino acid sequence as set forth in any one of SEQ ID NOs: 3-7, 12, 13, 22-24, or a combination thereof,
thereby optimizing treatment of a subject afflicted with a disease or disorder associated with amyloid beta accumulation.
